(19) United States
(12) Patent Application Publication
(10) Pub. No.: US 2010/0183561 A1
(43) Pub. Date: Jul. 22, 2010

(54) Biological therapeutic compositions and methods thereof

(75) Inventors: Ramasamy Sakthivel, Solon, OH (US); Donald J. Brown, Hopkinton, MA (US); Yukang Zhao, Framingham, MA (US); Adam Sorkin, Cleveland, OH (US); Vincent Pompili, Bexley, OH (US); Kevin Mcintosh, Brooklyn Park, MN (US)

Correspondence Address: MINTZ LEVIN COHN FERRIS GLOVSKY & POPEO, ONE FINANCIAL CENTER, BOSTON, MA, 02111, US

(73) Assignee: ARTERIOCYTE MEDICAL SYSTEMS, Cleveland, OH (US)

(21) Appl. No.: 12/459,829

(22) Filed: Jul. 7, 2009

Related U.S. Application Data
(60) Provisional application No. 61/134,120, filed on Jul. 7, 2008.
(60) Provisional application No. 61/199,187, filed on Nov. 12, 2008.

(51) Int. Cl. A61K 35/12; A61K 35/16

(52) U.S. Cl. 424/93.7; 424/530

(57) ABSTRACT

Novel uses of Platelet Rich Plasma (PRP) for infection prevention, bone repair and transmyocardial vascularization is disclosed. The present disclosure is directed to methods for preparing concentrated mesenchymal or haematopoietic stem cells, and autologous platelet-rich plasma (PRP) from the blood utilizing the Magallen® System. The stem cells and/or PRP may be combined with secondary biological agents such as antibiotics, fibrinogen and thrombin, and appropriately used in variety of medical conditions, such as, cardiovascular, thoracic, transplantation, head and neck, oral, gastrointestinal, orthopedic, neurosurgical, and plastic surgery.

### CROSS-REFERENCE TO RELATED APPLICATIONS

This application claims the benefit of priority to U.S. Provisional Application No. 61/134,120, filed Jul. 7, 2008, and U.S. Provisional Application No. 61/199,187, filed Nov. 12, 2008, and incorporates their disclosures herein by reference in their entireties.

### FIELD OF THE DISCLOSURE

The present disclosure is related to delivery systems for biological therapeutic compositions, methods of making thereof, and methods of using.

### BACKGROUND

When the lining of a blood vessel is damaged, a complex series of events takes place which is designed to prevent blood loss and, ultimately, to restore the integrity of the vessel. Although short-lived vasoconstriction and physical factors, such as pressure of extruded blood on the vessel wall, may play some part in haemostasis, the main factors in the haemostatic mechanism are platelets and the blood coagulation system.

Blood coagulation is the result of the complex interaction of a number of protein clotting factors through a cascade. In general, damage to the vascular endothelium exposes subendothelial structures, which attract platelets and induce them to aggregate reversibly. The protein thrombin, formed during activation of the coagulation pathway generates insoluble cross-linked fibrils of the protein fibrin and causes the platelets to aggregate irreversibly. The resulting platelet-fibrin clot is an effective barrier against loss of blood from the vascular system and also serves as a scaffold for subsequent repair of the lining of the blood vessel.

Bioadhesive sealants and fibrin glues represent a relatively new technological advance that duplicates the biological process of the final stage of blood coagulation. Clinical reports document the utility of fibrin glue in a variety of surgical fields, such as, cardiovascular, thoracic, transplantation, head and neck, oral, gastrointestinal, orthopedic, neurosurgical, and plastic surgery. At the time of surgery, the two primary components comprising the fibrin glue, fibrinogen and thrombin, are mixed together to form a clot. The clot adheres to the necessary tissues, bone, or nerve within seconds, but is then slowly reabsorbed by the body in approximately 10 days by fibrinolysis. Important features of fibrin glue are its ability to: (1) achieve haemostasis at vascular anastomoses particularly in areas which are difficult to approach with sutures or where suture placement presents excessive risk; (2) control bleeding from needle holes or arterial tears which cannot be controlled by suturing alone; and (3) obtain haemostasis in heparinized patients or those with coagulopathy. (See, e.g., Borst, H. G., et al., J. Thorac. Cardiovasc. Surg., 84:548-553 (1982); Walterbusch, G. J, et al., Thorac Cardiovasc. Surg., 30:234-235 (1982); and Wolner, F. J, et al., Thorac. Cardiovasc. Surg., 30:236-237 (1982)).

Despite the effectiveness and successful use of fibrin glue by medical practitioners in Europe, neither fibrin glue nor its essential components fibrinogen and thrombin are widely used in the United States. In large part, this stems from the 1978 U.S. Food and Drug Administration ban on the sale of commercially prepared fibrinogen concentrate made from pooled donors because of the risk of transmission of viral infection, in particular the hepatitis-causing viruses such as HBV and HCV (also known as non-A, non-B hepatitis virus). In addition, the more recent appearance of other lipid-enveloped viruses such as HIV, associated with AIDS, cytomegalovirus (“CMV”), as well as Epstein-Barr virus, and the herpes simplex viruses in fibrinogen preparations make it unlikely that there will be a change in this policy in the foreseeable future. For similar reasons, human thrombin is also not currently authorized for human use in the United States. Bovine thrombin, which is licensed for human use in the United States is obtained from bovine sources, which do not appear to carry significant risks for HIV and hepatitis, although other bovine pathogens, such as bovine spongiform, encephalitis, may be present.

There have been a variety of methods developed for preparing fibrin glue. For example, U.S. Pat. No. 4,627,879 to Rose et al., (hereinafter, “Rose”) discloses a method of preparing a cryoprecipitated suspension containing fibrinogen and Factor XIII useful as a precursor in the preparation of a fibrin glue which involves: (a) freezing fresh plasma from a single donor such as a human or other animal, e.g., a cow, sheep or pig, which has been screened for blood transmitted diseases, e.g., one or more of syphilis, hepatitis or acquired immune deficiency syndrome at about −80° C. for at least about 6 hours, preferably for at least about 12 hours; (b) raising the temperature of the frozen plasma, e.g., to between about 0° C. and room temperature, so as to form a supernatant and a cryoprecipitated suspension containing fibrinogen and Factor XIII; and (c) recovering the cryoprecipitated suspension. The fibrin glue is then prepared by applying a defined volume of the cryoprecipitate suspension described above and applying a composition containing a sufficient amount of thrombin, e.g., human, bovine, ovine or porcine thrombin, to the site so as to cause the fibrinogen in the suspension to be converted to the fibrin glue which then solidifies in the form of a gel.

A second technique for preparing fibrin glue is disclosed in, for example, U.S. Pat. No. 5,607,694 to Marx (hereinafter, “Marx”). Essentially a cryoprecipitate as discussed previously serves as the source of the fibrinogen component and then Marx adds thrombin and liposomes. A third method discussed by, for example, Berruyer, M. et al., “Immunization by Bovine Thrombin Used with Fibrin Glue During Cardiovascular Operations,” J. Thorac. Cardiovasc. Surg., 105(5):892-897 (1992), discloses a fibrin glue prepared by mixing bovine thrombin not only with human coagulant proteins, such as fibrinogen, fibronectin, Factor XIII, and plasminogen, but also with bovine aprotinin and calcium chloride.

The above patents by Rose and Marx, and the technical paper by Berruyer, et al. each disclose methods for preparing fibrin sealants; however, each of these methods suffer disadvantages associated with the use of bovine thrombin as the activating agent. A serious and life threatening consequence associated with the use of fibrin glues comprising bovine thrombin is that patients have been reported to have a bleeding diathesis after receiving topical bovine thrombin. This complication occurs when patients develop antibodies to the bovine factor V in the relatively impure bovine thrombin preparations. These antibodies cross-react with human factor V, thereby causing a factor V deficiency that can be sufficiently severe to induce bleeding and even death. (See, e.g., Rapaport, S. I., et al., Am. J. Clin. Pathol., 97:84-91 (1992); Berruyer, M., et al., J. Thorac. Cardiovasc. Surg., 105:892-897 (1993); Zehnder, J., et al., Blood, 76(10):2011-2016 (1990); Muntean, W., et al., Acta Paediatr., 83:84-7 (1994); Christine, R. J., et al., Surgery, 127:708-710 (1997)).

Another disadvantage associated with the methods disclosed by Marx and Rose is that the cryoprecipitate preparations require a large time and monetary commitment to prepare. Furthermore, great care must be taken to assure the absence of any viral contaminants.

A further disadvantage associated with the use of fibrin glues disclosed by Marx and Rose is the individual, to whom the fibrin glue is applied, is separately treated with antibiotics or other biologic agents to prevent or control infections. The antibiotics are generally administered using an intravenous injection of an antibiotic directly into the individual's blood stream. This injection increases the individual's overall systemic antibiotic concentration, but dilutes the actual dosage of antibiotic administered to the wound site susceptible to infection. When antibiotics are administered using via intravenous injections, a higher volume of antibiotic is required, thereby increasing costs to the patient.

A further disadvantage associated with administration of intravenous antibiotics is the actual release of the antibiotics in the individual's system generally results in “peaked” concentration levels as the antibiotics are administered to the patient.

Another disadvantage associated with the methods previously disclosed is that while human thrombin is contemplated for use as an activator, human thrombin is not available in the United States.

Bioadhesive sealants and fibrin glues have achieved moderate success in the treatment of wounds, such as surgical wounds. This is especially true where wounds include disruption or trauma to bone or muscle tissue.

Fracture of a long bone associated with crushing or structural muscles can often trigger acute compartment syndrome. Although fasciotomy is a useful treatment, delay or insufficient treatment leads to irreversible injury to the muscles, nerves, blood vessels, and bones. There is no option for conventional therapy in cases of poor blood supple in an injured leg. Restoration of bioactivity in the fractured site is thought to be essential for treatment of a non-union bone fracture. It is well known that bone fracture healing requires a blood supply, so bone marrow MNCs implantation might be useful not only for revascularization but also for subsequent bone regeneration. Battlefield injuries from shrapnel, grenade detonation, and fire often result in significant tissue, muscle, and dermal layer loss for military personnel. This trauma is often so severe that it impairs blood flow to point where it may result in the need for amputation. Furthermore, these injuries, much like those associated with motor vehicle accidents, and related fracture of a long bone are often associated with crushing of skeletal muscles and disruption of blood flow that triggers acute compartment syndrome. Within the muscle compartment, swelling and/or bleeding creates pressure on capillaries and nerves. When the pressure in the compartment exceeds the blood pressure within the capillaries, the capillaries collapse. This disrupts the blood flow to muscle and nerve cells that begin to die within hours without a steady supply of oxygen and nutrients. Unless the pressure is relieved quickly, this can cause permanent disability. Unlike the acute condition, chronic compartment syndrome is characterized by pain and swelling caused by exercise. It can be a significant problem for the patient.

Fasciotomy is a currently viable treatment for acute compartment syndrome, but delayed or insufficient treatment leads to irreversible injury to the muscles, nerves, blood vessels, and bones. There is often no option for conventional therapy in cases of poor blood supply to an injured leg. However, it is believed that restoration of bioactivity within the fracture site is essential for the successful treatment of severe and/or non-union fractures. Angiogenesis (new vessel formation) plays a key role in this process by enhancing remodeling of the traumatized bone. Experimental studies indicate that induction of angiogenesis by growth factors, cytokines, and stem or progenitor cells can contribute to regeneration of bone tissue and repair of the fractures. Additionally, recent studies indicate that autologous bone marrow mononuclear cell implantation increases collateral vessel formation in ischemic limb models and in patients, and may represent a viable treatment strategy for traumatic injury.

Techniques for cranioplasty and related craniofacial procedures have improved significantly in recent years, but obtaining graft material for repair of trauma or defects remains a significant challenge for reconstructive surgeons. The Centers for Disease Control and Prevention (CDC) estimated that over 22,000 cranioplasties were performed in 2005, along with an additional 30,000 facial fixations and 20,000 general craniotomies/craniectomies. These procedures often cost tens of thousands of dollars to perform, and associated ICU/ancillary care costs following surgery are estimated at $12,000 for a typical patient (Young et al. 2000). The total burden this care places on the health care system can exceed $1 billion annually. There is a need for novel bone replacement techniques that facilitate more rapid osteointegration and osteoconduction recovery, reduce complications, and alleviate the substantial associated costs of care.

In addition to the significant surgical and post surgical care costs, over $1.3 billion was spent in the US on bone graft materials alone in 2006; by 2013, it is estimated that $3.3 billion will be spent in 2013 (Frost et al. 2007). 6%, or about $200 Million in 2013 will be spent on craniofacial defect graft materials alone (DeFrances et al. 2007). Bone grafts and substitutes are among the most routinely employed surgical materials, used to facilitate secondary osteogenesis in craniofacial procedures, as well as long bone repair, spinal repair, and other orthopedic and oral/maxillofacial procedures (Lane et al. 1999). Bone autograft is an ideal material for craniofacial procedures, but it is available in limited quantity and the associated donor site morbidity is undesirable.

The bone substitute segment of this market comprises resorbable polymers, and bioactive or surface reactive ceramics and bioglasses which obviate many of the problems associated with allograft (infection, immune response) and autograft (donor site morbidity, limited donor material). These products represent only about 3% of bone substitute market expenditures. An additional 3% of bone graft related expenditure is derived from platelet rich plasma therapies. These treatments are used to deliver highly concentrated immune and wound healing cells, growth factors, and adhesive elements at the surgical site, and can be readily combined with bone grafts to accelerate the healing recovery (Frost et al. 2007). Both avenues for bone tissue repair are becoming more accepted by the reconstructive, neuro- and orthopedic surgeons responsible for repairing craniofacial and other bone defects. Current areas of experimentation suggest that repair strategies that incorporate elements of both (bioactive compounds and regenerative stem cells) may represent an effective, economical treatment that could improve outcomes, reduce recovery time, as well as minimize chance of post-operative complications that are expensive to treat and pose significant health risks to patients.

Calcium phosphate bone cements are used as bone substitutes because they are similar in chemical composition to the mineral component of bone. Hydroxyapatite (HA) cement was developed by the American Dental Association for maxillofacial surgery in 1986 (Jackson et al. 2000), and was approved for use by the FDA in 1996 (Hitchon et al. 2001). HA cement is very similar in structure to native bone mineral; in addition to bone apatite contains 4-6% carbonate in which results in somewhat different properties than pure HA (Constantz et al. 1995). It readily integrates with native bone, and can be readily prepared at the surgical site under physiological conditions (Costantino et al. 1991; Friedman et al. 1991). These properties have made it an appealing alternative graft material for cranial reconstruction (cranioplasty) as it is available in more abundant quantities than the ‘ideal’ calvarial bone autograft (and without added donor site morbidity) (Silva et al. 2005), and avoids a number of biocompatibility issues associated with non-resorbable polymethylmethacrylate bone cement (Hitchon et al. 2001).

Recent findings suggest, however, that HA has a number of drawbacks that may limit its efficacy in cranial reconstruction and other orthopedic procedures. First, though the material is highly biocompatible, and readily osseointegrates, the degree to which it is replaced by stable new bone is questionable (Ambard et al. 2006; Rupprecht et al. 2003; Lew et al. 1997). Additionally, there have been a number of reports of high incidences of post-operative infection that may be precipitated by an inflammatory reaction to cement fragments (Baker et al. 2002; Durham et al. 2003; Matic et al. 2004; Verheggen et al. 2001; Verret et al. 2005; Zins et al. 2007). Finally, the mechanical properties of hydroxyapatite are inferior to cortical bone, and not sufficient for certain applications (Matic et al. 2004; Ni et al. 2006).

More recently developed “next generation” calcium phosphate bone cements have improved upon HA cements by including a small fraction of carbonates. These cements, such as the Norian Cranial Reconstruction System (CRS) contain 5% carbonate and mimic key properties of bone apatite more closely than HA (such as solubility/resorbability). They demonstrate substantial promise as bone graft substitutes for craniofacial applications. CRS has recently been shown to produce, in some situations, vascularized, woven bone. However, though they outperform HA cements, it remains unclear how well these next generation products encourage regrowth of bone; bone auto- or allo-graft often demonstrate better long term performance. Research to better understand these products is ongoing.

Though not preferred when suitable biological graft material is available, titanium has been used in a number of forms over the past forty years in cranioplasty to provide adequate impact resistance and protection for brain associated soft tissues. Recently, titanium has been used to enhance reconstruction of cranial defects with novel bone graft and bone substitute materials. Hydroxyapatite cements have been the most common by far. However, work with plate-rich plasma and other novel avenues for bone regeneration have had promising results in maxillofacial reconstruction and may represent solutions that can overcome issues faced by more common bone cements.

Wounds, such as surgical wounds, are often associated with infections. Accordingly, treatment of wounds also involve measures to prevent infection. According to the Center for Disease Control report, the total number of postoperative surgical site infections (“SSI”) is estimated to be approximately 500,000 per year, among an estimated total of 27 million surgical procedures annually (see, e.g., C J DeFrances, K A Cullen, and L J Kozak, “National Hospital Discharge Survey: 2005 Annual Summary With Detailed Diagnosis and Procedure Data”, in Vital Health Statistics, 2007, National Center for Health Statistics), and account for approximately one quarter of the estimated 2 million nosocomial infections in the United States each year (see, e.g., D. S. Young, B. S. Sachais, and L. C. Jefferies, “The costs of disease”, Clin. Chem., 2000. 46(7), p. 955-66). These postoperative surgical site infections remain a major source of illness and a less frequent cause of death in the surgical patient (see, e.g., T. Jackson and R. Yavuzer, “Hydroxyapatite cement: an alternative for craniofacial skeletal contour refinements”, Br. J. Plast. Surg., 2000. 53(1), p. 24-9). Infections can result in longer hospitalization and higher costs.

Surgical site infection is one of the foremost concerns post surgery. According to the Centers for Disease Control and Prevention (“CDC”), in American hospitals alone, healthcare-associated infections account for an estimated 1.7 million infections and 99,000 associated deaths each year. Infections have significantly increased morbidity, mortality, and cost of patient care in the past 5 years, with hospitalization costs ranging from $3,000 to more than $30,000 per infection. (See, e.g., B. R. Constantz, et al., “Skeletal repair by in situ formation of the mineral phase of bone”, Science, 1995, 267(5205), p. 1796-9). Recent advancements in spinal surgery implants have resulted in their prevalent usage in diverse vertebral pathologies and, as a consequence, the associated post-operative infection rates have increased. The rate of postoperative infections is approximately 1% in elective spine surgery; however, when stabilizing metal implants are used, post-surgical infection rates have been reported from 2.1% to as high as 12%. (see, e.g., P. W. Hitchon, et al., “Comparison of the biomechanics of hydroxyapatite and polymethylmethacrylate vertebroplasty in a cadaveric spinal compression fracture model”, J. Neurosurg., 2001. 95(2 Suppl), p. 215-20).

These costs and the length of hospital stay are undoubtedly lower today for most patients based on improved surgical techniques and wound care. However, major complications such as deep wound infections seen in cardiac and spinal surgery continue to significantly increase the duration of hospitalization (as much as 20-fold) and the cost of hospitalizations (up to fivefold) (see, e.g., T. Jackson and R. Yavuzer, “Hydroxyapatite cement: an alternative for craniofacial skeletal contour refinements”, Br. J. Plast. Surg., 2000. 53(1), p. 24-9). Any surgical site infection after open heart surgery results in a substantial net loss of reimbursement to the hospital compared with uninfected cases, a factor that should motivate hospitals to continue to focus on implementing strategies to minimize the incidence of postoperative infections.

Recent advancements in spinal surgery have promoted the prevalent use of rigid titanium or stainless steel implants, which have been shown to increase the rate of infection from 1% to a range of 2.1% to 12%. (see, e.g., P. W. Hitchon, et al., “Comparison of the biomechanics of hydroxyapatite and polymethylmethacrylate vertebroplasty in a cadaveric spinal compression fracture model”, J. Neurosurg., 2001. 95(2 Suppl), p. 215-20). Surgical site infections have significantly increased morbidity, mortality and cost of patient care over the past years, with hospitalization costs ranging from $3,000 to $30,000 per infection. Staphylococcus aureus is the most common microorganism found in most hospital infections. (see, e.g., P. D. Costantino, et al., “Hydroxyapatite cement. I. Basic chemistry and histologic properties. Arch Otolaryngol Head Neck Surg”, 1991, 117(4), p. 379-84). With the contemporary use of systemic or intravenous anti-bacterial antibiotic therapy, only moderate levels of infection reduction have been achieved. A contributing factor to the increased infection rate has been the inability to prevent the eradication of infection due to failure in delivering sustained release of antibiotics at the surgical site post operatively. (see, e.g., C. D. Friedman, et al., “Hydroxyapatite cement. II. Obliteration and reconstruction of the cat frontal sinus. Arch Otolaryngol Head Neck Surg, 1991. 117(4): p. 385-9). Alternative delivery methods, such as Hydrogel, and various other biomaterials for sustained drug delivery have been explored (see, e.g., R. V. Silva, et al., The use of hydroxyapatite and autogenous cancellous bone grafts to repair bone defects in rats. Int J Oral Maxillofac Surg, 2005. 34(2): p. 178-84), however, systemic or intravenous antibiotics delivery still remains the most common technique of prophylactic post-surgical infection prevention.

Increasing rates of infection clearly define the lack of adequate prevention and treatment of surgical site infections (see, e.g., J. Ambard and L. Mueninghoff, “Calcium phosphate cement: review of mechanical and biological properties”, J Prosthodont, 2006, 15(5), p. 321-8; S. Rupprecht, et al., “Hydroxyapatite cement (BoneSource) for repair of critical sized calvarian defects—an experimental study”, J Craniomaxillofac Surg, 2003, 31(3), p. 149-53; D. Lew, et al., “Repair of craniofacial defects with hydroxyapatite cement”, J Oral Maxillofac Surg, 1997, 55(12), p. 1441-9; discussion 1449-51; S. B. Baker, et al., “Applications of a new carbonated calcium phosphate bone cement: early experience in pediatric and adult craniofacial reconstruction”, Plast Reconstr Surg, 2002, 109(6), p. 1789-96; S. R. Durham, J. G. McComb, and M. L. Levy, “Correction of large (>25 cm(2)) cranial defects with ‘reinforced’ hydroxyapatite cement: technique and complications”, Neurosurgery, 2003, 52(4), p. 842-5; discussion 845). Thus, there is a need for better method and system of preventing and treating surgical site infections.

There remains a need for developing improved procedures with treating any wound site and for preventing the development of infection. There remains a need for developing improved procedures with treating surgical wound sites and for preventing the development of infection of surgical wounds. Such wounds include postoperative surgical wounds such as wounds that result from crainioplasty. There remains a need for a convenient and practical method for preparing a bioadhesive sealant composition wherein the resulting bioadhesive sealant poses a zero risk of disease transmission and a zero risk of causing an adverse physiological reaction. Further, there is a need for a bioadhesive sealant that can be prepared to prevent infection at the wound site without increasing the overall systemic concentration increase of antibiotics.

Cell therapy has shown promising early results for cardiac repair following myocardial infarction (MI), both in animal and clinical trials (Freyman et al. 2006; Kocher et al. 2001; Orlic et al. 2001; Patel et al. 2005; Patel et al., 2007; Wollert et al. 2004). In particular, adult bone-marrow derived mesenchynal stem cells (MSCs) have been shown to have remarkable paracrine and regenerative properties when delivered following an MI (Freyman et al. 2006). One of the limitations in this therapy is the poor survival of the engrafted cells; in a pig model of MI, direct myocardial injection of allogeneic MSCs results in 97% cells loss by 14 days (Freyman et al. 2006).

The infarcted myocardium has poor vascular supply and an active inflammatory process, and thus it may fail to provide a viable microenvironment to help support the injected cells. The question then becomes: Is there means by which treatment of the scar tissue can lead to more favorable microenvironment for cardiac cell therapy? Transmyocardial revascularization (TMR) is a process by which channels are created into ischemic myocardium by either laser or mechanical means to enhance myocardial perfusion, by directly supplying tissue with endoventricular oxygenated blood or angiogenesis (Horvath et al. 1997; Li et al. 2003). In animals, TMR is known to upregulate VEGF, bFGF along with other growth factors, and induce at least a limited degree of angiogenesis using either mechanical or laser treatment (Chiotti et al. 2000; Horvath et al. 1999; Pelletier et al. 1998). Most studies have found a similar angiogenic response between mechanical versus laser TMR, except one where greater angiogenesis with laser versus mechanical TMR was reported (Mack et al. 1997).

A number of clinical trials have demonstrated successful treatment of angina using laser TMR (Allen et al. 1999; Horvath et al. 1997); however, the exact mechanism of action in humans has not been firmly established. Mechanical TMR has been used in small-animal studies, demonstrating enhanced response to cell therapy (Wang et al. 2006; Wang et al. 2006). However, the issue in translating these findings to a clinically relevant model is that in small animals there is significant perfusion of the ventricular wall via active diffusion due to thin myocardium. Using TMR as pretreatment to cell therapy may improve the microenvironment to enhance cell retention and long-term engraftment in a large-animal model of MI. TMR may act synergistically with the paracrine factors secreted by the engrafted MSCs, leading to a more potent effect on myocardial remodeling following an MI.

TMR is a treatment aimed at improving blood flow to areas of the heart that were not treated by angioplasty or surgery. A special carbon dioxide (CO₂) laser is used to create small channels in the heart muscle, improving blood flow in the heart. TMR is a surgical procedure. The procedure is performed through a small left chest incision or through a midline incision. Frequently, it is performed with coronary artery bypass surgery, but occasionally it is performed independently.

Once the incision is made, the surgeon exposes the heart muscle. A laser handpiece is then positioned on the area of the heart to be treated. A special high-energy, computerized carbon dioxide (CO₂) laser, called the CO₂ Heart Laser 2, is used to create between 20 to 40 one-millimeter-wide channels (about the width of the head of a pin) in the oxygen-poor left ventricle (left lower pumping chamber) of the heart. The doctor determines how many channels to create during the procedure. The outer areas of the channels close, but the inside of the channels remain open inside the heart to improve blood flow.

The CO₂ Heart Laser 2 uses a computer to direct laser beams to the appropriate area of the heart in between heartbeats, when the ventricle is filled with blood and the heart is relatively still. This helps to prevent electrical disturbances in the heart.

Clinical evidence suggests blood flow is improved in two ways: 1) The channels act as bloodlines. When the ventricle pumps or squeezes oxygen-rich blood out of the heart, it sends blood through the channels, restoring blood flow to the heart muscle. 2) The procedure may promote angiogenesis, or growth of new capillaries (small blood vessels) that help supply blood to the heart muscle.

Throughout this description, including the foregoing description of related art, any and all publicly available documents/publications described herein, including any and all U.S. patents, are specifically incorporated by reference herein in their entirety. The foregoing description of related art is not intended in any way as an admission that any of the documents described therein, including pending United States patent applications, are prior art to embodiments of the present disclosure. Moreover, the description herein of any disadvantages associated with the described products, methods, and/or apparatus, is not intended to limit the disclosed embodiments. Indeed, embodiments of the present disclosure may include certain features of the described products, methods, and/or apparatus without suffering from their described disadvantages.

### SUMMARY

Delivery systems, therapeutic compositions, and methods are provided for the treatment of a wound, such as a surgical, and for the prevention of infection to the wound site. The body's natural healing process may be stimulated at the cellular level using autologous platelet gel (APG), to treat open wounds and may be beneficial in preventing infection, reducing pain, blood loss and bruising.

A delivery system and a method for preparing a biological therapeutic and applying it to an individual are provided. The biological therapeutic may be prepared by isolating a biological fluid where the biological fluid can be inactive platelet rich plasma or stem cells. The biological fluid may be isolated using a blood centrifuge, combining the biological fluid with an activating agent and a biological agent to prepare a biological therapeutic and applying the therapeutic to an individual.

Autologous platelet gel compositions are provided to stimulate new bone formation in bone defects such as but not limited to cranial defects. In some embodiments, autologous platelet gel compositions with derived mononuclear cells and/or bone morphogenetic proteins are provided to stimulate new bone formation in bone defects such as but not limited to cranial defects.

Autologous platelet gel compositions are provided for therapeutic angiogenesis and osteogenesis for use in but not limited to patients with traumatic bone injuries.

Autologous platelet gel compositions with autologous bone marrow mononuclear cells are provide for therapeutic angiogenesis and osteogenesis for use in but not limited to patients with traumatic bone injuries.

According to some embodiments, methods are provided for developing a novel autologous platelet-based sustained-release system for antibiotic delivery to any wound site. This treatment may be configured to significantly reduce the incidence of infection. Although the principle function of platelets is homeostasis, these cells also have important functions in antimicrobial host defense.

In some embodiments, the methods include the steps of stimulating body's natural healing at a cellular level using autologous platelet gel (“APG”) to treat open wounds. The present disclosure's method may also be beneficial in preventing infection, and reducing pain, blood loss and bruising.

According to some embodiments, delivery systems are provided for autologous bioadhesive sealant compositions, and more particularly to a convenient and practical method for preparing a bioadhesive sealant. In some embodiments, the bioadhesive sealant may be prepared from blood components derived from the patient, who may receive the bioadhesive sealant, and combining them with a biologic agent to form an autologous bioadhesive therapeutic sealant.

According to some embodiments, drug delivery mechanisms are provided where autologous platelet rich plasma (PRP) may be combined with general antibiotics to provide a sustained release of antibiotics directly to an open wound site to prevent or fight infection. Stem cells may be isolated from Umbilical Cord Blood (UCB) CD133 cells to induce neovascularization in wound healing. The PRP can be used to prevent infection and neovascularization in wound healing.

According to some embodiments, transmyocardial laser revascularization (TMR) plus an intramyocardial injection of platelet rich plasma or stem cells derived from the bone marrow may be used to provide angina relief.

Accordingly, it is an object of this disclosure to provide a method for preparing a completely autologous bioadhesive sealant composition.

Another object of the present disclosure is to provide an autologous bioadhesive sealant composition with derived mononuclear cells and/or bone morphogenetic proteins to stimulate new bone formation in bone defects.

An additional object of the present disclosure is to provide an autologous bioadhesive sealant composition with derived mononuclear cells and/or bone morphogenetic proteins to stimulate new bone formation in cranial defects.

Additional objects, advantages, and novel features of this disclosure shall be set forth in part in the description that follows, and in part will become apparent to those skilled in the art upon examination of the following specification or may be learned by practice of the disclosure. The objects and advantages of the disclosure may be realized and attained by means of the instrumentalities, combinations, compositions, and methods particularly pointed out in the appended claims.

### BRIEF DESCRIPTION OF THE DRAWINGS

For a better understanding of the present disclosure, reference is made to the following description, taken in conjunction with the accompanying drawings, in which like reference characters refer to like parts throughout.

FIG. 1. Magellan® Autologous Platelet Gel (APG) System. The Magellan® system is a one-source biologic concentrator, consisting of a microprocessor-controlled centrifuge and syringe pumps.

FIG. 2. The figure depicts the preparation of autologous platelet rich plasma using the Magellan® System. The Magellan® system collects and concentrates platelets and white blood cells from a small volume of a patients blood.

FIG. 3. Customizing of platelet concentration depending on patient need.

FIG. 4A. Cell yield after Magellan® separation of peripheral blood.

FIG. 4B. Cell yield after Magellan® separation of cord blood.

FIG. 5. Immediate aggregation of PRP into a solid following combination with Vancomycin solution.

FIG. 6. Antibiotic enriched PRP. Left to right (all final antibiotic concentrations=75 ug/ml): 1. 6 mL PRP+6 mL Thrombin (1 kU/ml)/Vancomycin (833 μg/mL) in 10% Calcium chloride solution. 2. 6 mL PRP+6 mL Vancomycin (833 μg/mL) only in 10% Calcium chloride solution. 3. 6 mL PRP+6 mL Thrombin (1 kU/ml)/Cefazolin (833 μg/mL) in 10% Calcium chloride solution. 4. 6 mL PRP+6 mL Cefazolin Cefazolin (833 μg/mL) only in 10% Calcium chloride solution.

FIG. 7A) shows a Phoenix™ combined TMR and cell delivery system. The center channel is for a laser fiber. Three needles with multiple side holes are also shown. FIG. 7B) shows a Phoenix™ combined TMR and cell delivery system used for cell delivery into myocardium or scar depicting radial dispersion of cells to minimize leakage.

FIGS. 8A-I. Histological analysis of myocardial tissue after TMR and cell treatment, with relevant controls.

FIG. 9. High-power (100×) TMR+ Cells. Single arrow demonstrates round mononuclear or inflammatory cell. Double arrow depicts a mesenchymal stem cell.

FIG. 10. (A) Cell viability. (B-D). The echocardiography of all animals throughout the course of the study that received all three treatments. (B) The end diastolic volume increased after infarction at 2 weeks but did not continue to increase after treatment. (C) The overall ejection fraction as measured by echocardiography decreased after infarction but did not continue to decrease after treatment. (D) The end diastolic diameter increased after infarction but actually decreased 1 week after treatment at termination.

### DETAILED DESCRIPTION

For the purposes of promoting an understanding of the principles of the disclosure, reference will now be made to the embodiments illustrated in the drawings, and specific language will be used to describe the same. It should nevertheless be understood that no limitation of the scope of the disclosure is thereby intended, such alterations and further modifications in the illustrated device, and such further applications of the principles of the disclosure as illustrated therein being contemplated as would normally occur to one skilled in the art to which the disclosure relates are also encompassed by the disclosure.

Autologous Platelet-Rich Plasma (PRP) is produced from blood, by removing red blood cells through apheresis based centrifugation, yielding a high concentration of platelets, immune cells, and endogenous growth factors. PRP enhances natural healing processes, and may reduce or prevent infections. It is appropriately used in surgical applications, including cardiothoracic, neurologic, has become increasingly accepted for use in orthopedic surgery. Recent studies have suggested that PRP improves the outcomes of surgical procedures, such as total knee arthroplasty by acting upon the surgical wound (Berghoff et al. 2006; Everts et al. 2006). However, there is a growing body of research that suggests PRP also enhances osteogenesis through several mechanisms that speed the remodeling and repair process (Wrotniak et al. 2007). Thor, et al., have demonstrated that bone grafts treated with PRP produce bone at an increased rate (Thor et al. 2007). Others have found that PRP significantly enhanced bone healing. It can also enhance distraction osteogenesis for craniofacial procedures (Kinosita et al. 2008). Though activated PRP effects osteogenesis directly by improving differentiation and bone morphogenetic protein (BMP) expression (Wiltfang et al. 2004; Schlegel et al. 2004; Slapnicka et al. 2008), a vascularization of graft material has been shown to be a secondary effector of osteogenic activity (Yokota et al. 2008).

PRP has also been used successfully as a delivery medium to provide BMP, other osteogenic factors, and cells in healing bone. Lin et al. have demonstrated that the concentrated growth factors associated with PRP induces osteoblastic differentiation (Lin et al. 2006). It has been shown that PRP can serve as an effective, rate-controlled delivery system for TGF-1 and IGF. In particular, platelet gel enhances osteogegesis when used in conjunction with bone morphogenetic proteins (BMP-2 and 7) (Jansen et al. 2005; Tomoyasu et al. 2007). In addition to growth factors, Yamada et al were successful in delivering mesenchymal stem cells to repair maxillofacial defects in an animal model (Yamada et al. 2004). These methods represent a promising alternative to use of traditional repair materials that are associated with additional patient morbidity (autograft), infection (allograft/demineralized bone products), and poor osteoconduction (calcium phosphate cements).

### Bioadhesive

In some embodiments, the present disclosure is directed to a method for preparing a bioadhesive composition and combining the composition with a biologic agent and delivering the composition to an individual.

In other embodiments, the present disclosure is directed to a bioadhesive composition wherein the risks associated with the use of bovine and recombinant human thrombin are eliminated.

In other embodiments, the present disclosure is directed to an autologous bioadhesive sealant composition or fibrin glue prepared by a two-phase method, wherein all of the blood components for the bioadhesive sealant are derived from a patient to whom the bioadhesive sealant will be applied.

Additional advantages and novel features of this disclosure shall be set forth in part in the description that follows, and in part will become apparent to those skilled in the art upon examination of the following specification or may be learned by the practice of the disclosure. The objects and advantages of the disclosure may be realized and attained by means of the instrumentalities, combinations, compositions, and methods particularly pointed out in the appended claims.

To achieve the foregoing and in accordance with the purposes of the present disclosure, as embodied and broadly described therein, the disclosure provides methods for forming or preparing an autologous bioadhesive sealant. These methods include the steps of forming a platelet rich plasma (“PRP”) or platelet poor plasma (“PPP”) containing an anticoagulant. The platelet rich plasma or platelet poor plasma may then be divided into two portions and the first portion is restored so that it can coagulate, thus forming a clot. The clot may be triturated and the resulting serum may be collected. The bioadhesive sealant composition is then prepared by combining a defined volume of the second portion of platelet rich plasma or platelet poor plasma with a sufficient volume of serum causing the fibrinogen in the second portion of platelet rich plasma or platelet poor plasma to be converted to fibrin, which then solidifies in the form of a gel.

In some embodiments, the present disclosure is directed to the methods described above further including drawing whole blood from a patient, and adding an inactivating agent to the blood. In some aspects of the disclosure, the method further includes a step of combining a lyophilized biologic agent with the inactivated whole blood to form a biologic therapeutic, (“Therapeutic”) which is then spun to agitate the components. The Therapeutic is combined with an anticoagulant agent and is then separated to form PRP and PPP using a blood centrifuge. The PRP is isolated and combined with an activating agent. The activated Therapeutic is then applied to the individual from whom the blood was drawn.

According to further embodiments, the method for preparing autologous bioadhesive sealant further comprises converting fibrinogen in the second portion of at least one of the PRP and the PPP to fibrin; forming a gel from the fibrin. In more specific embodiments, the method further comprises adding an inactivating agent to the whole blood.

In yet more specific embodiments, the method further comprises combining the inactivated blood with a lyophilized biological agent; forming a biological therapeutic agent.

In other embodiments, the method of forming PRP or PPP further comprises combining the biological therapeutic agent with the anticoagulant; and separating the combination to form the PRP or PPP. According to specific embodiments the combination of PRP and biological therapeutic agent is separated using a centrifuge.

According to some embodiments, the method of preparing bioadhesive sealant further comprises isolating the PRP or PPP; combining the isolated PRP or PPP with an activating agent; and applying the activated biological therapeutic agent to the patient.

In more specific embodiments, the lyophilized biologic therapeutic agent may be added during separation of the whole blood into PRP and PPP. Optionally, the activating agent is thrombin. Preferably, the activating agent may be lyophilized thrombin.

Some embodiments of the methods of the disclosure include drawing whole blood from an individual, and adding an inactivating agent to the blood. The whole blood may then be separated to form PRP and PPP using a blood centrifuge. During the separation process a lyophilized biologic therapeutic is added to the PRP either during separation or collection of the PRP into a collection syringe used in conjunction with the blood centrifuge. The PRP combined with a biologic agent forms a Therapeutic. The Therapeutic may be combined with an activating agent. The Therapeutic combined with the activating agent is then applied to the individual from whom the whole blood was drawn.

Other embodiments of the methods of the disclosure include drawing whole blood from an individual, and adding an inactivating agent to the blood. The whole blood may then be separated to form PRP and PPP using a blood centrifuge. The PRP may be combined with an activating agent and a lyophilized biologic agent and applied to an individual from whom the whole blood was drawn. Optionally, the activating agent may be autologous thrombin. Preferably, the activating agent may be lyophilized bovine thrombin. More preferably, the activating agent may be lyophilized recombinant thrombin.

In some embodiments of the disclosure, the biologic agent may be selected from a wide variety of drugs or proteins with other biologic activities may be added to the platelet rich plasma of phase-two. Examples of the agents to be added to the platelet rich plasma prior to the addition of the serum include, but are not limited to, analgesic compounds, antibacterial compounds, including bactericidal and bacteriostatic compounds, antibiotics (e.g., adriamycin, erythromycin, gentimycin, penicillin, tobramycin or vancomycin), antifungal compounds, anti-inflammatories, antiparasitic compounds, antiviral compounds, enzymes, enzyme inhibitors, glycoproteins, growth factors (e.g. lymphokines, cytokines), hormones, steroids, glucocorticosteroids, immunomodulators, immunoglobulins, minerals, neuroleptics, proteins, peptides, lipoproteins, tumoricidal compounds, tumorstatic compounds, toxins and vitamins (e.g., Vitamin A, Vitamin E, Vitamin B, Vitamin C, Vitamin D, or derivatives thereof). It is also envisioned that selected fragments, portions, derivatives, or analogues of some or all of the above may be used.

In some embodiments, the biologic agent may be lyophilized. Preferably, the lyophilized biologic agent may be an antibiotic. In certain specific embodiments, the lyophilized biologic agent may be selected from a group of antibiotics comprising: adriamycin, erythromycin, gentimycin, penicillin, tobramycin, vancomycin, cefazolin, and classes of antibiotics including, but not limited to: thamoxicillin, cephalosporins, macrolides, fluoroquinolones, sulfonamides, tetracyclines and aminoglycosides. In certain preferred embodiments, the lyophilized antibiotic may be vancomycin. In certain preferred embodiments, the lyophilized antibiotic may be cefazolin.

In some embodiments, growth factors may be selected from: lymphokines and cytokines.

According to further embodiments, vitamins may be selected from: Vitamin A, Vitamin E, Vitamin B, Vitamin C, Vitamin D, and derivatives thereof.

In some embodiments of the present disclosure, the PRP, as discussed above, can be configured to include concentrated stem cells. In some embodiments, the PRP can be configured to include concentrated stem cells with or without concentrated platelets.

In instances where the desired bioadhesive sealant composition of the present disclosure is to further function as a delivery device of drugs and proteins with other biologic activities the method of the present disclosure may be modified as follows. The disclosure couples the use of Autologous Platelet Gel (“APG”) generated to reduce infections associated with surgery, hospital related infections and open wound infections, with a biologic therapeutic agent, such as antibiotics. In some embodiments, APG provides a scaffold for the sustained release of the antibiotic, extending the localized antimicrobial environment at the surgical site, which will reduce the incidence of post surgical infection.

In some embodiments, the present disclosure can be configured to use Arteriocyte Medical System, Inc.'s MAGELLAN® (a biologic concentrator and a delivery MAGELLAN® System) autologous platelet system to generate APG. The MAGELLAN® system is disclosed in co-owned patents, such as, U.S. Pat. Nos. 6,444,228, 6,596,180, 6,719,901, 6,830,762, 6,899,813, 6,942,639, and applications, such as, U.S. patent application Ser. Nos. 10/848,302, 11/004,023, 11/044,984, 11/049,010, 11/115,076, 11/120,305, 11/159,482, the disclosures of which are incorporated herein by reference in their entireties.

The MAGELLAN® System is an automated fully closed system and includes a microprocessor-controlled centrifuge and syringe pumps, that concentrates platelets, neutrophils, white blood cells and mononuclear cells, and delivers consistent, reproducible platelet concentrate. The MAGELLAN® System also provides a technology platform for autologous platelet rich fibrin sealants and other biotechnology applications. The MAGELLAN® System can also be used to separate and concentrate a variety of cells, including stem cells, endothelial cells, and leukocytes. MAGELLAN® was designed to be used at the point of care or in the clinical laboratory for the safe and rapid preparation of PRP and PPP from a small sample of blood.

The present disclosure can also be configured to use MAGELLAN® System to collect and concentrate platelets and white blood cells from a small volume of a patient's own blood. PRP may be automatically and quickly separated from anticoagulated whole blood and dispensed into a separate sterile syringe. PRP may be mixed with calcified thrombin in the MAGELLAN® Ratio Dispenser Kit, thereby creating APG. APG is a rich source of growth factors.

In some embodiments, the present disclosure can be configured to use another platelet system separator to generate APG. In certain specific embodiments, the present disclosure can also be configured to combine an antibiotic with autologous platelet rich plasma gel for targeted delivery into an open wound for the sustained release of antibiotic treatment to prevent infection. In some embodiments, Vancomycin is the antibiotic combined with PRP. In some other embodiments, cefazolin is the antibiotic combined with PRP. In certain preferred embodiments, the present disclosure can also be configured to have another antibiotic combined with PRP, wherein another antibiotic, includes but not limited to, any penicillin or amoxicillin, cephalosporins, macrolides, fluoroquinolones, sulfonamides, tetracyclines and aminoglycosides.

In some embodiments, the present disclosure can be configured to include a multiple-site implant device for delivering a controlled release delivery system for antibiotics using autologous platelet rich plasma. This configuration prevents infection growth via an application of a system for extended release of localized antibiotic delivery in an infected wound site. Optionally, the infected wound may be a surgical site. Preferably, the surgical site may be located on the human body. Optionally, the surgical site may be located on an animal's body. In certain embodiments, the present disclosure can be configured to use PRP in combination with antibiotics to prevent infection, to fight an existing infection, or to prevent a relapse of infection in an open wound.

The present disclosure may also be directed to a single continuous antibiotic delivery a system and a method for providing a constant antimicrobial action locally at the surgical site. The some embodiments, the method includes delivering a lower dose of antibiotic directly at the infection site versus systemic or intravenous delivery and reducing side effects associated with higher dose systemic delivery. APG-antibiotic may be configured to be applied directly to the wound site prior to the closing of the wound or incision. The combination of platelets and antibiotic action is configured to produce an advantageous condition to extend the antimicrobial environment. This method further reduces the incidence of surgical site infection, open wound infection, hospital-related infection and morbidity, reduces hospitalization costs, and improves clinical outcomes.

According to the disclosure, PRP can also be mixed with autograft and/or allograft bone prior to application to an orthopedic site. PPP and PRP can also be made from a mixture of blood and bone marrow. The disclosure is not limited to using the MAGELLAN® System, but discloses it as one of the modes in light of its efficiency, ease of use, and effectiveness is separating blood into its components. Stem cells may be isolated using the MAGELLAN® System (or equivalent) to deliver stem cells. Stem cells are obtained by combining bone marrow and whole blood and using the blood centrifuge to isolate the stem cells.

The present disclosure also provides systems and methods to obtain stem cells from an individual, and combine the stem cells with a biological agent to form a Stem Cell Therapeutic and applying it to the individual from whom the stem cells were obtained. In some embodiments, the biological agent is selected from a wide variety of drugs or proteins with other biological activities and may be added to the platelet rich plasma of phase-two. Examples of the agents to be added to the platelet rich plasma prior to the addition of the serum include, but are not limited to, analgesic compounds, antibacterial compounds, including bactericidal and bacteriostatic compounds, antibiotics (e.g., adriamycin, erythromycin, gentimycin, penicillin, tobramycin), antifungal compounds, anti-inflammatories, antiparasitic compounds, antiviral compounds, enzymes, enzyme inhibitors, glycoproteins, growth factors (e.g. lymphokines, cytokines), hormones, steroids, glucocorticosteroids, immunomodulators, immunoglobulins, minerals, neuroleptics, proteins, peptides, lipoproteins, tumoricidal compounds, tumorstatic compounds, toxins and vitamins (e.g., Vitamin A, Vitamin E, Vitamin B, Vitamin C, Vitamin D, or derivatives thereof). It is also envisioned that selected fragments, portions, derivatives, or analogues of some or all of the above may be used.

In some embodiments, the biological agent is lyophilized. In certain specific embodiments, the lyophilized biological agent is an antibiotic. Preferably, the lyophilized biologic agent may be selected from a group of antibiotics comprising: penicillin or amoxicillin, cephalosporins, macrolides, fluoroquinolones, sulfonamides, tetracyclines or aminoglycosides. More preferably, the lyophilized antibiotic may be vancomycin or cefazolin.

### Autologous Platelet Gel

This disclosure couples the use of APG generated to reduce infections associated with surgery, hospital related infections and open wound infections. APG provides a scaffold for the sustained release of the antibiotic, extending the localized antimicrobial environment at the surgical site, which will reduce the incidence of post surgical infection.

One embodiment of the disclosure combines an antibiotic with autologous platelet rich plasma gel for targeted delivery into an open wound for the sustained release of antibiotic treatment to prevent infection. Preferably, vancomycin and/or cefazolin is the antibiotic combined with PRP. In other embodiments another antibiotic may be combined with PRP including but not limited to any penicillin or amoxicillin, cephalosporins, macrolides, fluoroquinolones, sulfonamides, tetracyclines and aminoglycosides.

In other embodiments of the disclosure intra cardiac blood is extracted at periodic intervals to evaluate antibiotic levels.

The disclosure also provides for a controlled release delivery system for antibiotics using autologous platelet rich plasma may be delivered using a multiple-site implant device. This system prevents infection growth via the application of a system for extended release of localized antibiotic delivery in an infected wound site. Optionally, the infected wound is a surgical site. Preferably, the surgical site is located on any part of the human body. Optionally, the surgical site is located on any animal's body.

In other embodiments of the disclosure PRP is used in combination with antibiotics for infection prevention, to fight an existing infection or to prevent a relapse of infection in an open wound.

The disclosure also provides an autologous bioadhesive sealant composition, comprising a combination of a predetermined volume of a portion of at least one of a platelet rich plasma (“PRP”) and a platelet poor plasma (“PPP”) with a sufficient volume of a serum, wherein said combination is prepared by obtaining whole blood from a patient; forming the PRP or the PPP from the whole blood, wherein the PRP or PPP can be configured to contain an anticoagulant; separating each one of the PRP and the PPP into a first portion and a second portion; restoring the first portion of at least one of the PRP and the PPP, thereby forming a clot; triturating the clot and collecting the serum; combining a predetermined volume of the second portion of at least one of the PRP and the PPP with a sufficient volume of the serum to form the combination. According to further embodiments, preparation of the combination comprises converting fibrinogen in the second portion of at least one of the PRP and the PPP to fibrin; forming a gel from the fibrin.

The disclosure also provides methods in which an inactivating agent is added to the whole blood, and a lyophilized biological agent is combined with the inactivated blood to form a biological therapeutic agent.

According to some embodiments formation of the PRP or the PPP includes combining the biological therapeutic agent with the anticoagulant; and separating the combination of the biological therapeutic agent and the anticoagulant to form the PRP or PPP, where the combination is separated using a centrifuge.

According to other embodiments the PRP or the PPP are isolated; and the isolated PRP or PPP may be combined with an activating agent; and the activated biological therapeutic agent may be applied to the patient. The lyophilized biologic therapeutic agent may be added during separation of the whole blood into PRP and PPP.

According to some embodiments the lyophilized biologic therapeutic agent may be added during collection of the PRP or PPP into a container. Also may be added is an activating agent such as thrombin, where the activating agent is lyophilized thrombin.

According to some embodiments the PRP or the PPP are combined with another antibiotic.

According to some embodiments of the disclosure, a stem cell therapeutic composition, comprises combining the stem cells obtained from a patient with a biological agent, where the composition is applied to the patient using a syringe.

The biological agent added to the stem cell composition may be selected from: analgesic compounds, antibacterial compounds, including bactericidal and bacteriostatic compounds, antibiotics, antifungal compounds, anti-inflammatories, antiparasitic compounds, antiviral compounds, enzymes, enzyme inhibitors, glycoproteins, growth factors, hormones, steroids, glucocorticosteroids, immunomodulators, immunoglobulins, minerals, neuroleptics, proteins, peptides, lipoproteins, tumoricidal compounds, tumorstatic compounds, toxins and vitamins, or fragments, portions, derivatives, or analogues thereof.

The antibiotics included in the stem cell composition may be from a group consisting of: adriamycin, erythromycin, gentimycin, penicillin, tobramycin, vancomycin, cefazolin, amoxicillin, cephalosporins, macrolides, fluoroquinolones, sulfonamides, tetracyclines and aminoglycosides.

In some embodiments, a continuous antibiotic delivery system for constant antimicrobial action locally at the surgical site is provided. In some such embodiments, the continuous antibiotic delivery system is a single continuous antibiotic delivery system. For example, this method delivers a lower dose of antibiotic directly at the infection site versus systemic or intravenous delivery and may reduce the side effects associated with higher dose systemic delivery. APG-antibiotic will be applied directly to the wound site prior to the closing of the wound or incision. The combination of platelets and antibiotic action produces an advantageous condition to extend the antimicrobial environment. This method reduces the incidence of surgical site infection, open wound infection, hospital-related infection and morbidity and reduce hospitalization costs and improve clinical outcomes.

One embodiment of the disclosure includes using the Magellan® System, the biologic concentrator and delivery system noted earlier in the present disclosure. The system my include a microprocessor-controlled centrifuge and syringe pumps. The Magellan® System collects and concentrates platelets and white blood cells from a small volume of a patient's own blood. Platelet Rich Plasma (PRP) is automatically and quickly separated from anticoagulated whole blood and dispensed into a separate sterile syringe. PRP is mixed with calcified thrombin in the Magellan® Ratio Dispenser Kit, thereby creating Autologous Platelet Gel (APG). APG is a rich source of growth factors.

Aspects of the Magellan® System concentrates platelets, neutrophils, white blood cells and mononuclear cells, and delivers consistent, reproducible platelet concentrate. The Magellan® System also provides a technology platform for autologous platelet rich fibrin sealants and other biotechnology applications. The Magellan® System can also be used to separate and concentrate a variety of cells, including stem cells, endothelial cells and leukocytes. Magellan® was designed to be used at the point of care or in the clinical laboratory for the safe and rapid preparation of Platelet Rich Plasma (PRP) and Platelet Poor Plasma (PPP) from a small sample of blood. PRP can also be mixed with autograft and/or allograft bone prior to application to an orthopedic site. PPP and PRP can also be made from a mixture of blood and bone marrow. The disclosure is not limited to using the Magellan® System, but discloses it as the best mode because of its efficiency, ease of use, and effectiveness is separating blood into its components. Stem cells may be isolated using the Magellan® System (or equivalent) to deliver stem cells from Umbilical Cord Blood (UCB) CD133 cells to induce neovascularization in wound healing.

Patients with medically refractory angina that are not amenable to conventional revascularization may be injected with platelet rich plasma while undergoing a minimally invasive, robatic-assisted sole Transmyocardial laser revascularization to improve angina. The autologous platelet rich plasma contains angiogenic growth factors, that in connection with TMR substantially relieve the effects of angina. The autologous PRP may be produced using a point of care platelet separator such as Arteriocyte's Magellan® System. The angiogenic up regulation of injured myocytes by the laser becomes a fertile area for an enhanced stem cell paracrine effect. (Reyes et al., and Umemura et al.).

To achieve the foregoing and other objects and in accordance with the purposes of the present disclosure, as embodied and broadly described therein, the method of this disclosure comprises the formation of an autologous bioadhesive sealant composition comprising the steps of forming a platelet rich or platelet poor plasma containing an anticoagulant. The platelet rich or platelet poor plasma is then divided into two portions. The first portion is restored using a restorative agent, allowing it to coagulate thus forming a clot. The clot is then triturated and the resulting serum is collected. The mononuclear cells or bone morphogenetic proteins are added to either serum or the second portion of plasma. The bioadhesive sealant composition is then prepared by combining a defined volume of the second portion of platelet rich or platelet poor plasma with a sufficient volume of serum causing the fibrinogen in the second portion of platelet rich or platelet poor plasma to be converted to fibrin, which then solidifies in the form of a gel. Another object of the present disclosure is to use autologous bone marrow derived from mononuclear cell therapy for revascularization of limb extremities to promote muscle tissue regeneration in acute trauma and chronic muscle injuries.

Another object of the present disclosure is to use an autologous bioadhesive sealant composition with autologous bone marrow derives from monocuclear cell therapy for revascularization of limb extremities to promote muscle tissue regeneration in acute trauma and chronic muscle injuries.

The treatment of bone wounds such as traumatic injury from shrapnel or grenade detonation would treat current military personnel with acute battlefield wounds and veterans who have chronic Compartment syndrome and extreme circulatory problems due to past injuries. The long term patient management and cost savings derived from potential prevention of limb amputations as a result of chronic lack of peripheral blood flow will potentially provide a more effective therapeutic intervention to improve the healing, workload stress capacity, and long term viability of tissues injured due to trauma on the battlefield.

### Stem-Cell Therapeutics

The disclosure also provides a method for preparing a stem cell therapeutic composition may comprise: obtaining stem cells from a patient; combining the stem cells with a biological agent to form a stem cell therapeutic composition, then applying the stem cell therapeutic composition to the patient. In certain specific embodiments, the biological agent may be selected from any one or more of: analgesic compounds, antibacterial compounds, including bactericidal and bacteriostatic compounds, antibiotics, antifungal compounds, anti-inflammatories, antiparasitic compounds, antiviral compounds, enzymes, enzyme inhibitors, glycoproteins, growth factors, hormones, steroids, glucocorticosteroids, immunomodulators, immunoglobulins, minerals, neuroleptics, proteins, peptides, lipoproteins, tumoricidal compounds, tumorstatic compounds, toxins and vitamins. The antibiotics may be any one or more of: adriamycin, erythromycin, gentimycin, penicillin, tobramycin and vancomycin. And the growth factors may be: lymphokines and/or cytokines.

In some embodiments, vitamins that are used may be any one or more of: Vitamin A, Vitamin E, Vitamin B, Vitamin C, Vitamin D, and derivatives thereof.

According to further embodiments of the disclosure, the lyophilized biological agent can be configured to include fragments, portions, derivatives, or analogues of analgesic compounds, antibacterial compounds, including bactericidal and bacteriostatic compounds, antibiotics, antifungal compounds, anti-inflammatories, antiparasitic compounds, antiviral compounds, enzymes, enzyme inhibitors, glycoproteins, growth factors, hormones, steroids, glucocorticosteroids, immunomodulators, immunoglobulins, minerals, neuroleptics, proteins, peptides, lipoproteins, tumoricidal compounds, tumorstatic compounds, toxins and vitamins.

According to specific embodiments, the lyophilized biological agent may be an antibiotic, selected from any one or more of: penicillin, amoxicillin, cephalosporins, macrolides, fluoroquinolones, sulfonamides, tetracyclines and aminoglycosides. Preferably, the lyophilized biological agent may be vancomycin or cefazolin.

In some embodiments, the disclosure provides for stem cell therapeutic composition may be administered to a mammal for treatment of medical conditions, selected from the group consisting of: cardiovascular, thoracic, transplantation, head and neck, oral, gastrointestinal, orthopedic, neurosurgical, and plastic surgery.

### Methods for Drug Delivery

According to some embodiments of the disclosure, methods are provided for preparing the combination comprising converting fibrinogen in the second portion of at least one of the PRP and the PPP to fibrin; forming a gel from the fibrin.

According to some embodiments of the disclosure, a drug delivery mechanism, comprises: an autologous platelet rich plasma (PRP); general antibiotics; wherein a combination of the autologous platelet rich plasma (PRP) and the general antibiotics may be configured to provide a sustained release of antibiotics directly to an open wound site to prevent or fight infection.

The composition, according to some embodiments of the disclosure, further comprises: an autologous platelet gel; derived mononuclear cells and/or bone morphogenetic proteins; wherein a composition of the autologous platelet gel and the derived mononuclear cells and/or bone morphogenetic proteins is configured to stimulate new bone formation in bone defects such as but not limited to cranial defects.

The composition according to some embodiments of the disclosure, further comprises: an autologous platelet gel; autologous bone marrow mononuclear cells; wherein a composition of the autologous platelet gel and the autologous bone marrow mononuclear cells may be configured to be used for therapeutic angiogenesis and osteogenesis for use in but not limited to patients with traumatic bone injuries.

The disclosure also provides a continuous antibiotic delivery system for constant local antimicrobial action at a surgical site, comprises a delivery mechanism for delivering a lower dose of antibiotic directly at an infection site configured to reduce side effects associated with a higher dose systemic delivery; the delivery mechanism may be configured to apply an APG-antibiotic directly to a wound site prior to closing of a wound or an incision; wherein a combination of platelets and antibiotic action may be configured to produce an advantageous condition to extend the antimicrobial environment and further configured to reduce an incidence of surgical site infection, open wound infection, hospital-related infection, morbidity, reduce hospitalization costs, and improve clinical outcomes.

According to further embodiments, the continuous antibiotic delivery system may be further configured to couple the use of generated APG to reduce infections associated with surgery, hospital related infections and open wound infections; where APG provides a scaffold for the sustained release of the antibiotic, extending the localized antimicrobial environment at the surgical site and configured to reduce the incidence of post surgical infection. The system further comprises a platelet system separator to generate APG.

According to further embodiments, the continuous antibiotic delivery system may be configured to combine an antibiotic with autologous platelet rich plasma gel for targeted delivery into an open wound for the sustained release of antibiotic treatment to prevent infection. An example of such a combination is when Vancomycin is combined with PRP. Other antibiotics that may be combined are penicillin or amoxicillin, cephalosporins, macrolides, fluoroquinolones, sulfonamides, tetracyclines and aminoglycosides.

According to some embodiments of the disclosure, the continuous antibiotic delivery system is configured to evaluate antibiotic levels at periodic intervals using an intra cardiac blood extraction, where the system is configured to controllably release delivery system for antibiotics using autologous platelet rich plasma using a multiple-site implant device.

The continuous antibiotic delivery system may also be configured to prevent infection growth via the application of a system for extended release of localized antibiotic delivery in an infected wound site, such as surgical site on any part of human or animal body.

According to some embodiments of the disclosure, the continuous antibiotic delivery system may be configured to use PRP in combination with antibiotics for infection prevention, to fight an existing infection or to prevent a relapse of infection in an open wound.

### Methods of Treatment

According to some embodiments of the disclosure, a method for treating ischemic cardiac disease with stem cells, may comprise (i) performing transmyocardial laser revascularization (TMR) to create microvascular environment into infarcted myocardial tissue; and (ii) implantation of said stem cells into said myocardial tissue; wherein the administration of TMR allows higher retention and survival of intramuscularly injected stem cells after TMR; where the stem cells are isolated from the group consisting of: placenta, adipose tissue, lung, bone marrow, umbilical cord, or blood. Examples of adult stem cells include: haematopoietic stem cells, mammary stem cells, mesenchymal stem cells, endothelial stem cells, neural stem cells, olfactory adult stem cells, and testicular stem cells.

The Magellan STEM-PREP System concentrates stem cells by centrifugally separating them from fluids and cells of other densities. This is analogous to the manner in which the Magellan System seperates and concentrates platelets/white blood cells from whole blood. The Magellan System dispenses bone marrow (or a mixture of blood and bone marrow) into centrifuge chambers for automatic, centrifugal separation and subsequent collection of an enriched cell population.

In addition to the components found in whole blood, bone marrow also contains HSC and MSC stem cells. HSC and MSC stem cells have a density similar to that of platelets and white blood cells. Centrifugal processing of bone marrow (or a bone marrow mixture) causes the stem cells to separate and be collected in the same fraction as the platelets and white blood cells. As a consequence, the stem cells are concentrated within the smaller collection volume.

According to further embodiments, TMR administration to said myocardial tissue leads to release of prothrombin or thrombin into said tissue.

According to some embodiments, the TMR involves a computerized laser to create small channels in the heart muscle, thereby increasing angiogenic growth factors in said laser injury site. The angiogenic growth factors may influence the survival of said intramuscularly injected stem cells after TMR.

In some embodiments, the number of channels created by TMR may be between 10-20, 20-25, 26-30, 31-35, 36-40, 41-45, or 20-40 approximately 1 mm wide channels. In some embodiments, the size of the channels may vary between less then 1 mm, 1.1 mm, 1.2 mm, 1.3 mm, 1.4 mm, 1.5-2.0 mm channels. In some preferred embodiment the size of the channels is 1 mm wide.

According to some embodiments, the stem cells may be mesenchymal stem cells.

According to some embodiments, the stem cells may be hematopoietic stem cells.

According to some embodiments, the stem cells may be prepared by a stem cell fractionation process to concentrate the mononucleated cells from the bone marrow, where the fractionation process comprises involves the Magellan® system.

According to some embodiments, the angiogenic growth factors may comprise: Fibroblast growth factors (FGF), Vascular endothelial growth factor (VEGF), VEGF receptors (VEGFR), Neuropilin 1 (NRP1), Angiopoietin 1 (Ang1), TEK tyrosine kinase (Tie2), platelet-derived growth factor (PDGF), Transforming growth factor beta (TGF-13), endoglin, Chemokine (C—C motif) ligand 2 (CCL2 or MCP-1), integrins, VE-cadherin, ephrin, plasminogen activators, plasminogen activator inhibitor-1, Nitric oxide synthases (NOS), and Cyclooxygenase-2 (COX-2).

The disclosure also provides a method for sustained or continuous release of antibiotics directly to an open wound site to prevent or fight infection comprising: providing a mixture of a predetermined amount of autologous platelet rich plasma (PRP) and providing a predetermined amount of at least one general antibiotic; where the combination of said autologous platelet rich plasma (“PRP”) and said general antibiotic enables sustained or continuous release of the antibiotic.

The method for sustained or continuous release of antibiotics, provided in the disclosure, involved an antibiotic combined with PRP, where the antibiotic is selected among: adriamycin, erythromycin, gentimycin, penicillin, tobramycin, vancomycin, cefazolin, amoxicillin, cephalosporins, macrolides, fluoroquinolones, sulfonamides, tetracyclines and aminoglycosides.

The method further provides that a lower dose of antibiotic is delivered directly at an infection site configured to reduce side effects associated with a higher dose systemic deliver; wherein the method is configured to apply the PRP-antibiotic directly to a wound site prior to closing of a wound or an incision.

The method as provided in the disclosure is configured to controllably release delivery system for the antibiotics using the PRP using a multiple-site implant device.

The disclosure also provides a method for stimulating new bone formation in bone defects in which an autologous platelet gel and a plurality of mononuclear cells, multinuclear cells, and/or bone morphogenetic proteins are applied to an area of bone defect. The composition of the autologous platelet gel and the mononuclear cells, multinuclear cells, and/or bone morphogenetic proteins stimulates new bone formation around the bone defect.

According to disclosure above, the multinuclear cells are bone-marrow derived osteoclasts; the bone morphogenic protein may be selected among the members in the BMP family of proteins: BMP1, BMP2, BMP3, BMP4, BMP5, BMP6, BMP7, BMP8a, BMP8b, BMP10, and BMP15; and the mononuclear cells may be bone-marrow derived mesenchymal stem cells (BM-MSCs) and/or bone-marrow derived mononuclear cells (BM-MNCs).

### DEFINITIONS

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In the case of conflict, the present specification, including definitions, will control. In addition, the materials, methods, and examples are illustrative only not intended to be limiting. Other features and advantages of the disclosure will be apparent from the following detailed description and claims.

For the purposes of promoting an understanding of the embodiments described herein, reference will be made to preferred embodiments and specific language will be used to describe the same. The terminology used herein is for the purpose of describing particular embodiments only, and is not intended to limit the scope of the present disclosure. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth. Thus, for example, a reference to “a host cell” includes a plurality of such host cells, and a reference to “an antibody” is a reference to one or more antibodies and equivalents thereof known to those skilled in the art, and so forth.

Stem cells stem cells are undifferentiated cells, found throughout the body after embryonic development, that multiply by cell division to replenish dying cells and regenerate damaged tissues. Also known as somatic stem cells, they can be found in juvenile as well as adult animals and humans. They may be isolated from the placenta, adipose tissue, lung, bone marrow, or blood. Examples of adult stem cells include: haematopoietic stem cells, mammary stem cells, mesenchymal stem cells, endothelial stem cells, neural stem cells, olfactory adult stem cells, and testicular stem cells.

Additional objects, advantages, and novel features of this disclosure shall be set forth in part in the description for the examples that follow, and in part will become apparent to those skilled in the art upon examination of the following or may be learned by the practice of the disclosure. The objects and the advantages of the disclosure may be realized and attained by means of the instrumentalities and in combinations particularly pointed out in the appended claims. The following examples are meant for illustration purposes only and not limiting to the embodiments of the present disclosure.

### EXAMPLES

### Example 1

### Magellan® Autologous Platelet Gel (APG) System

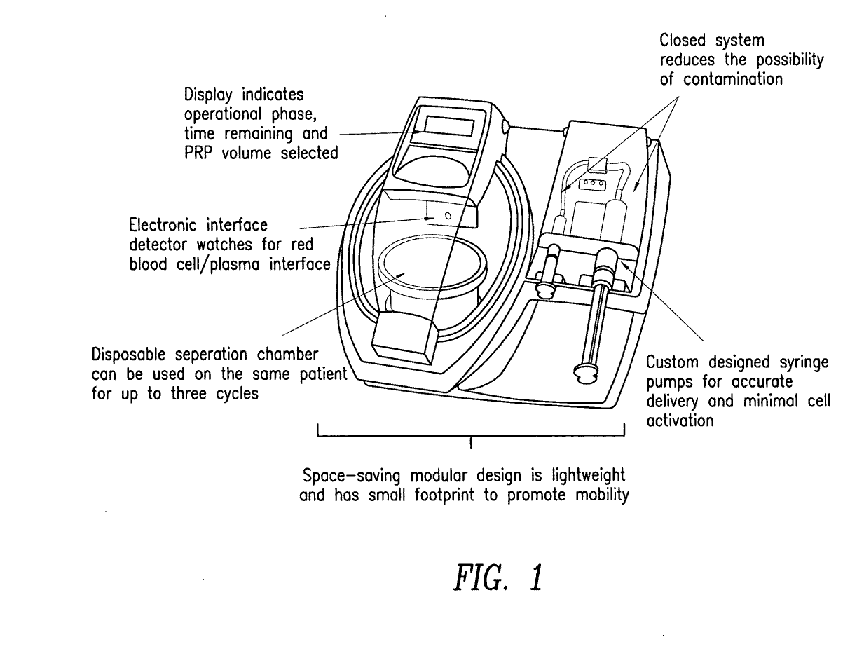

In surgical practice, accelerating the rate of wound healing and regenerating normal tissue function is desirable. Autologous concentrated platelet-plasma gel product was prepared for an adhesive advantage and to stimulate improved wound healing. The platelet rich plasma (PRP) was prepared automatically and quickly by Magellan® system (FIG. 1), which is a biologic concentrator and delivery Magellan® system, consisting of a microprocessor-controlled centrifuge and syringe pumps. The Magellan® system was used to concentrate platelets and white blood cells from a small volume of a patient's own blood, and was also used to concentrate mononucleated cells from bone marrow. Platelet Rich Plasma (PRP) was automatically and quickly separated from anti-coagulated whole blood, and dispensed into a separate sterile syringe. PRP was mixed with calcified thrombin in the Magellan® Ratio Dispenser Kit, thereby creating Autologous Platelet Gel (APG). APG is a rich source of growth factors. Table 1-1 discloses the test data showing that soft tissue growth factors are concentrated in PRP with the Magellan® System.

TABLE 1-1

| | Whole Blood | PRP |
|---|---|---|
| Growth Factor Mean ± S.D | (60 mL) | (6 mL) |
| PDGF-AB (ng/mL) | 10.2 ± 1.4 | 88.4 ± 28.8 |
| PDGF-AA (ng/mL) | 2.7 ± 0.5 | 22.2 ± 4.2 |
| PDGF-BB (ng/mL) | 5.8 ± 1.4 | 57.8 ± 36.6 |
| TGF-β1 (ng/mL) | 41.8 ± 9.5 | 231.6 ± 49.1 |
| VEGF (pg/mL) | 83.1 ± 65.5 | 597.4 ± 431.4 |
| bFGF (pg/mL) | 10.7 ± 2.9 | 48.4 ± 25.0 |
| EGF (pg/mL) | 12.9 ± 6.2 | 163.3 ± 49.4 |

The Magellan® system was originally developed to prepare a small amount of platelet concentrate, loaded into the machine, from a sample of blood taken by phlebotomy from a donor or patient into a 60 cc syringe. The device mechanically pushes the plunger attached to the syringe, forcing the anti-coagulated blood to flow into a separation chamber through its integrated tubing. The separation chamber is located on a centrifuge rotor plate. The tubing and centrifuge were constructed to allow flow into and out of the separation chamber during operation of the unit without a rotating seal (that is, using a closed system method). The platelet-rich plasma is separated from the red cells. The red cells are discharged back into the original 60 mL syringe, and the platelet-rich plasma may flow into a separate 10 mL syringe.

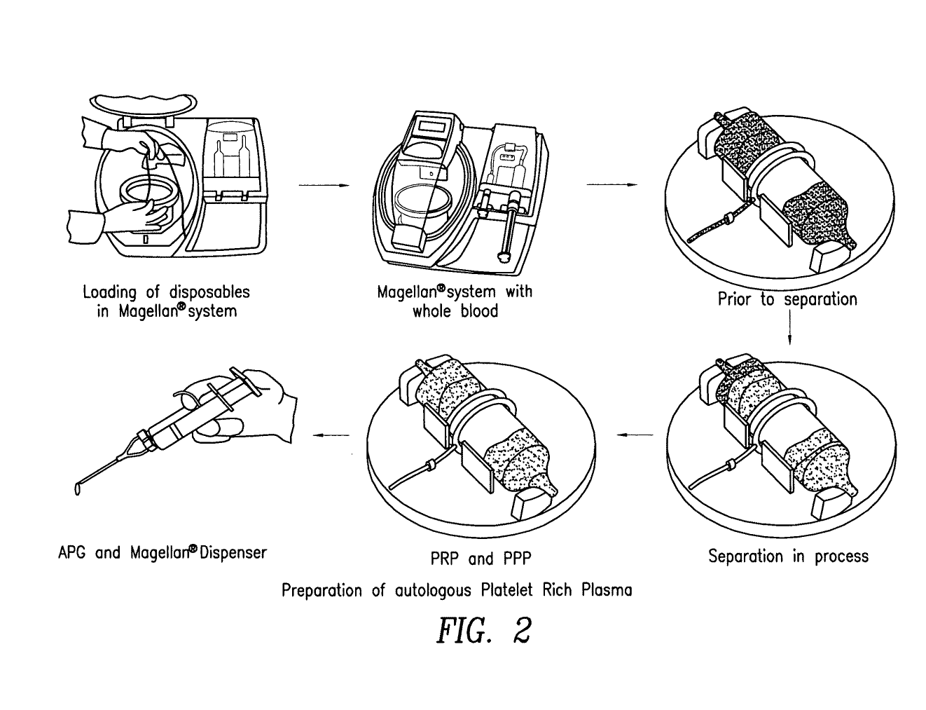

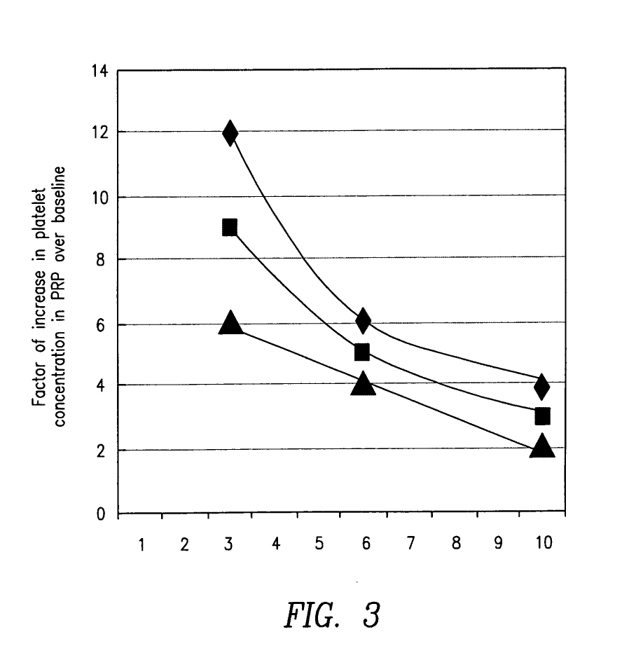

To prepare the Autologous Platelet Gel (APG), two 60-mL aliquots of anti-coagulated blood (13% anticoagulant citrate dextrose formula A) was obtained from each subject by venipuncture. Each aliquot was processed by an autologous platelet separator (Magellan® Autologous Platelet Separator, Arteriocyte Medical System) to yield 5 mL of PRP from each aliquot, thereby obtaining a total of 10 mL of PRP from each subject. One milliliter of PRP was used for platelet cell count analysis (Cell Dyn 1700 Henatology Analyzer; Abbott Diagnosticsm Abbott Park, Ill.). An autologous serum dispenser kit (Magellan® Autologous Serum Dispenser Kit: Arteriocyte Medical System) was used to create approximately 1.3 mL of autologous thrombin-rich serum from 2 mL of PRP (FIG. 2). The PRP was created at the wound site by co-dispensing the remaining PRP and the thrombin-rich serum using an autologous serum dispenser kit and a 5-cm cannula tip (Magellan® 2″ Cannula Tip; Arteriocyte Medical Systems). The platelet concentration can be customized depending on patient need as shown in FIG. 3.

The Magellan® System also concentrated autologous immune cells (white blood cells) more than 3 times over the baseline concentration in circulating blood. White blood cells are implicated in infection prevention. Table 1-2 discloses the blood cell yields using Magellan® System.

TABLE 1-2
Magellan Blood Cell Yields

| | | | Concentration |
|---|---|---|---|
| Mean ± S.D.; | Initial Blood | PRP | Levels |
| n = 5 | (60 mL) | (6 mL) | (×Baseline) |
| PLT | 184.9 ± 14.7 | 1356.4 ± 148.0 | 7.32 ± 0.4 |
| (×1000 μL) | | | |
| WBC | 5.18 ± 1.0 | 16.04 ± 6.3 | 3.2 ± 1.3 |
| (×1000 μL) | | | |
| Hct (%) | 39.5 ± 3.9 | 7.5 ± 1.6 | N/A |

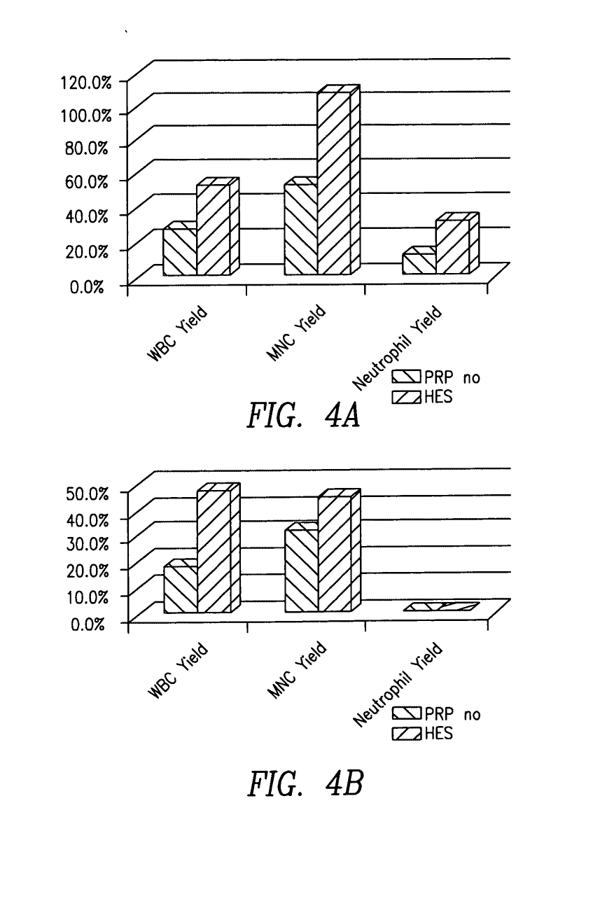

The Magellan® Autologous Platelet Rich Plasma concentration device was modified to provide an automated, fully closed, and sterile system (FIG. 2) for volume reduction of blood components and consistent high recovery of mononuclear cells (MNCs) from cord blood or bone marrow for cyropreservation purposes for the public and private cord blood banking use, providing improved cellular harvests for potential use in transplant medicine (FIG. 4).

Advantage of the Magellan® system: The Magellan® System is a fully automated closed loop processing system that requires limited intervention during processing, and provides reproducible platelet concentration yields. Other available systems require multiple sample transfers with manual operator intervention, increasing the chances of contamination. The Magellan® System is the only system available for platelet concentration that is based on blood bank developed pheresis-based technology. Competitive systems that use floating discs to separate the platelets, produce inconsistent results due to platelet's adherent affinity for the disc.

Summary: The Magellan® System is an automated fully closed system that concentrates platelets, neutrophils, white blood cells and mononuclear cells, and delivers consistent, reproducible platelet concentrate. The Magellan® System also provides a technology platform for autologous platelet rich fibrin sealants and other biotechnology applications. The Magellan® System can also be used to separate and concentrate a variety of cells, including stem cells, endothelial cells and leukocytes. Magellan® was designed for use at the point of care or at a clinical laboratory for the safe and rapid preparation of Platelet Rich Plasma (PRP) and Platelet Poor Plasma (PPP) from a small sample of blood. PRP can also be mixed with autograft and/or allograft bone prior to application to an orthopedic site. PPP and PRP can also be made from a mixture of blood and bone marrow.

TABLE 1-3

| | | Benefits of |
|---|---|---|
| Procedure | Description | platelet gel |
| Decubitis | Treatment of pressure sores on the | Wound healing |
| ulcers | skin and underlying tissues | Infection control |
| Venostasis | Ulcers on the skin and underlying | Wound healing |
| ulcers | tissues due to vascular abnormalities | Infection control |
| | | Angiogenesis |
| Diabetic foot | Ulcers developing from poor distal | Wound healing |
| ulcers | circulation in people with diabetes | Infection control |
| | | Angiogenesis |
| Spider bites | Wounds occurring from spider bites | Wound healing |
| | | Infection control |

### Example 2

### Release Kinetics of Vancomycin, Embedded with Autologous Platelet Rich Plasma Gel, During Targeted Delivery into a Surgical Wound

The study disclosed herein was designed to evaluate the efficiency of the Magellan® System generated platelet rich plasma (PRP) coupled with an antibiotic, for example Vancomycin, as a continuous controlled release system of antibiotic delivery, for the prevention of infection in a rabbit spinal implant infection model using Staphylococcus aureus infection. The study described in the Specific Aim 1 is designed to determine the release kinetics of Vancomycin when coupled with PRP.

Experimental approach: To decipher the basic mechanism of antibiotic release kinetics, experimental methods are designed where an antibiotic is coupled with Platelet Rich Plasma (PRP). Intracardiac blood extraction at periodic intervals, for the evaluation of antibiotic levels, is employed to follow the pharmacokinetics of drug delivery systems. HPLC-Concentration/time profile is used to understand the duration of antibiotic presence in the animal blood stream and hence the effect on release kinetics.

Experimental Methods: Preparation of Autologous Platelet Rich plasma Two 60-ml aliquots of anti-coagulated blood (13% anticoagulant citrate dextrose formula A) is obtained from blood donors after consenting. Each aliquot is processed by the autologous platelet separator (Magellan® Autologous Platelet Separator, Arteriocyte Medical System) to yield 5 mL of PRP from each aliquot, thereby obtaining a total of 10 mL of PRP from each subject.

Antimicrobial agents: Vancomycin 80, 160 and 320 g/L (Eli-Lilly, Indianapolis, Ind.) stock solutions is prepared according to the manufacturer's recommendations and is stored at −20° C. An appropriate concentration as described below is used for both in vitro and in vivo studies described. If the diluted Vancomycin reduces the tensile strength of the PRP preparation gel, an appropriate powder form (usually 15 mg/kg body weight), mixed with calcium chloride and thrombin solution before making the gel, is used. Additionally, a more diluted form of PRP gel with low tensile, may rapidly release the antibiotics from the gel, therefore alternative consideration as described above is necessary to overcome such an outcome.

In vitro elution studies: An autologous serum dispenser kit (Magellan® Autologous Serum Dispenser Kit; Arteriocyte, Inc) is used to create approximately 1.3 mL of PRP. Vancomycin 80, 160, and 320 g/L of Vancomycin, and approximately 1.3 mL of Thrombin rich serum is co-dispensed to create a 2 ml PRP gel using the serum dispenser kit, and two 5-cm cannula tips (Magellan® 2″ Cannula tip; Arteriocyte Medical Systems Inc). PRP is subjected to fives cycles of repeated freezing and thawing: at −25° C. for 2 h followed by half an hour at room temperature. The release of antibiotic by diffusion from PRP is removed and transferred to a tube containing 5 mL of fresh PBS. The elutes for each time point (0.5, 1, 2, 3, 4, 8, 12 and 24 hrs) is stored at −20° C. and assayed within 10 days. Each elution series is replicated six times. The above in vitro study is necessary to adjust tensile strength of PRP preparation to have optimal release of antibiotics delivery in an in vivo system.

Determination of the antibiotic concentration: The elution samples of PBS is assayed by agar diffusion microbiological assay. Indicator organisms is Bacillus subtilis ATCC 6633 for Vancomycin. Fifty milliliters of sterile nutrient agar (Antibiotika-Agar Nr.5; Merck, Germany) is seeded with the appropriate bacteria at a concentration of 10⁸ cfu/L and poured into 22 cm diameter round Petri dishes. The agar is allowed to set, and 14 wells (10 mm diameter) is punched into the agar and then filled with 100 μL of the elution samples and antibiotic calibrators. Calibrator concentrations are 0.625, 1.25, 2.5 and 5.0 mg/L, prepared in PBS. After incubation at 37° C. for 18 h, the zones of inhibition are read using a micrometer. The antibiotic concentration of elutes is determined by computer-assisted regression analysis. Each sample and calibrator is analyzed on two separate agar plates, giving two readings per sample, with the mean being taken for calculation.

In vivo antibiotic release kinetics study of PRP: 20 Male athymic rats (8- to 12-week-old) is purchased from Harlem (Harlem Sprague-Dawley, Indiana, USA). The animals is divided into four group of 5 rats/group (Group-1: Control (saline); Group-2: PRP alone injected; Group-3: Vancomycin alone; Group-4: Vancomycin and PRP together). Back and major part of the animal will shaved a day prior to surgery and the animal is anesthetized using 80 mg/kg of ketamine and 12 mg/kg of xylazine. An incision is made in the lumbar region of the spine, and the saline, Vancomycin or PRP+/−Vancomycin is injected in the region using the Magellan® PRP dispenser (Arteriocyte Medical System). Appropriate Volume (1-1.5 ml) of vancomycin in the concentration described above is used. The incision is closed using staplers (Ethicon, Inc). The entire procedure is carried out in a laminar flow hood and aseptic conditions will follow throughout the procedure. At scheduled times, 6 hr, 12 hr, 24 hr, 36 hr, 48 hr then 3 d, 5 d, 7 d, 10 d and 14 days, animals are anaesthetized and bled by intracardiac injection with a heparinized syringe. The study may be extended all the way up to 14 days to calculate any residual antibiotic remaining in circulation. The blood samples is centrifuged at 12,000 rpm for 4 min and the supernatant is analyzed in HPLC-C4 column using a gradient of MilliQ. The elute is monitored using an UV detector and reported as serum vancomycin detected.

The effects of PRP have been evaluated in many different ways, but with conflicting results. The lack of evidence for the efficacy of PRP from the first human and animal studies and low scientific value of many studies due to randomized studies have challenged researchers to leave human and animal studies for more basic research and in vitro experimental models. This specific aim evaluates the use of human PRP coupled with Vancomycin with the anticipation that the autologous platelet gel will consistently extend the continuous release of the coupled Vancomycin into the wound.

Several in vitro and in vivo studies have demonstrated the possibility that cancellous bone could be used as a carrier of antibiotics for local delivery. However, the release of antibiotics from the loaded cancellous bone is too rapid and uncertain: Further studies using demineralization of cancellous bone to increase the amount of antibiotic adsorbed, and coating of the freeze-dried antibiotic-impregnated cancellous bone with bio-compatible material has been suggested to prolong antibiotic release. It was found that significantly larger amounts of vancomycin were adsorbed into the demineralized bone matrix (DBM) than into the non-demineralized blocks. The blood coating was found to increase the duration of vancomycin release from the blocks. With demineralization and blood coating, the blocks eluted vancomycin higher than therapeutic concentration for a 5-week period. The potential use of DBM as a carrier for antibiotics is limited to clinical scenarios where the product is being utilized for bone healing and would not be considered as a first line approach to the majority of spine surgeries where bone incorporation is not required (laminectomy, discectomy, etc.). Similar studies have been reported by several investigators using a variety of other synthetic materials such as hydrogels, which have been reported to allow a sustained antibiotic release at surgical site wounds.

The purpose of the in vitro and in vivo experiments described in the specific aims are straight forward in understanding the kinetics of antibiotics release, which is important for the proposed in vivo rabbit infected spinal fusion model studies. The scaffold strength of the PRP will play a key role in controlling kinetics of the antibiotics release. Appropriate coupling of Vancomycin with PRP may not be achieved by simply mixing these two together for the purpose of sustained Vancomycin slow release. The use of demineralized cancellous bone with PRP may further improve sustained Vancomycin release. In addition, other scaffolding (both biological and non biological) technologies such as Hydrogel may be used to couple Vancomycin and PRP to fully utilize the novel autologous PRP gel for surgical site infection prevention.

### Example 3

### Controlled Sustained Release System for Antibiotics Using the Magellan® Autologous Platelet Rich Plasma in a Multiple-Site Implant Device

Experimental approach: We aim to investigate the prevention of infection with controlled antibiotic delivery system at a spinal surgical site. Although multiple experimental protocols have been established in order to study spinal infection, the use of New Zealand white rabbit has increased since the description of posterolateral intertransverse arthrodesis model by Boden et al. in 1995. A well-accepted established model on spinal infection is adopted for these series of studies.

Experimental Method: Animals: Twenty healthy New Zealand White Female Rabbits, each weighing approximately 2.5 to 3.0 kg. Prior to the surgery all the animals are acclimated to the new environment and fed standard diet. The animals are caged individually at constant temperature. The animals are randomly treated as follows: Control (bacterial inoculation only); PRP alone; local application of Vancomycin alone; or: Vancomycin coupled PRP.

Bacterial Inoculum: S. aureus (ATCC 33593, American Type Culture Collection, Rockville, Md.), originally isolated from a patient with septicemia, is used as the bacterial inoculums for the spinal rabbit infection model. For the preparation of the bacterial inoculum, the organism is typically grown in soy broth (TSB) at 37° C. for 18 h and is then diluted to the required concentration with sterile TSB prior to the application. Total bacterial count is determined using standard procedure of suspending the prepared dilutions of the inoculum on blood agar plate.

TABLE 3-1

| Randomization | SPINAL LEVELS | | |
|---|---|---|---|
| Schedule | T13 | L3 | L6 |
| N = 6 | PRP | Control | Vancomycin alone |
| N = 6 | Vancomycin alone | Control | Vancomycin/PRP |
| N = 6 | Vancomycin/PRP | Control | PRP |

Surgical Procedure: The entire back and entire gluteal region of the rabbits are shaved the day before the surgery. After 12 hours of fasting the animals are pre-medicated with butorphanol tartrate (0.1 mg/kg) and then anesthetized with intramuscular injection of 44 mg/kg of ketamine HCL, 5 mg/kg xylazine and 0.75 mg/kg acepromazine maleate half hour later. The positions of the desired vertebrae are marked on the back of the animal and 0.5-1.0 ml of bupivacaine (Marcaine; Sanofi Wintrop, New York, N.Y.) is injected subcutaneously at every site as local anesthesia. The entire animal is covered in sterile drapes exposing the marked regions for surgery.

Two rabbits are sacrificed and exsanguinated. The platelet concentrator system is utilized to prepare PRP that is utilized for the study. Eighteen study animals are then prepared for surgery. Three separate 3 cm incisions are made overlying the T13, L3 and L6 spinous processes. The fascia and muscle layers are maintained to preserve separation of each compartment and prevent cross-contamination. The spinous process at each level is excised, limited laminectomy performed and a stainless steel K-wire (0.875 mm) are then inserted across the transverse processes to simulate a spinal fixation device. Each site are then inoculated with 10³ CFU of the S. aureus strain. Each treatment level (T13 and L6) is randomized but the L3 level serves as the control (bacterial inoculation only) and no PRP or antibiotics added (See Table 2-1). PRP with or without vancomycin is prepared and added to the randomized levels. The fascia is closed tightly by using biodegradable Vicryl 2-0 (Ethicon Incorporation, NJ) after which the skin is closed using Ethilon nylon sutures 2-0 (Ethicon Inc, Somerville, N.J.).

The surgery is performed under strict sterile conditions in an aseptic hood and all the standard animal protocols are followed. After the surgery the animals are monitored closely, receive standard post operative pain medication (4 mg per kg of Carprophane, Rimadyl Vet, Pfizer Vericore Ltd, Dundee, UK) for 3 days. All the animals are housed individually in standard cases, provided with antibiotic free rabbit chow and monitored daily. They are closely observed for any signs of sepsis and body weight, temperature is recorded regularly.

Using an intravenous injection of 10 mg/kg of pentobarbital the animals are killed 8 days after the experiment. Prior to death, blood is drawn to determine presence of systemic sepsis under sterile conditions. Skin from the suture area, fascia, and muscle and from the wire implants of both transverse processes are removed to monitor systemic infection. The implants are sonicated (Ultrasonik 100; NEY, Bloomfield, Conn.) and the tissue samples are homogenized (model GLH; Omni International, Waterbury, Conn.) for 45 minutes to detach the bacteria. After serial plated dilutions on TSA, the samples are incubated for 24 hours at 37° C. Finally bacterial count for per gram tissue sample are conducted to determine the bacterial counts at the particular site. Two or more tissue sample from the same site are extracted to determine the implant centered infection.

The study described in this example is designed to understand whether continuous antibiotic delivery using Magellan® PRP at the implant-centered surgical infection site in a spinal fusion rabbit model may improve surgical site infection. Sustained antibacterial action at the surgical site may prevent the incidence of infection. The wound healing properties of the Platelet gel might speed recovery due to the presence of high levels of growth factors in Magellan® PRP. Investigations have shown the therapeutic efficacy of an antimicrobial peptide, human lactoferrin 1-11 (hLF1-11), in overcoming antibiotics resistance bacterial infection. Thus, alternative approaches to prevention of infection such as use of antimicrobial peptides (eg. human lactoferrin 1-11) to overcome bacterial antibiotic resistance with evaluations of the PRP delivery system may be incorporated in this system.

Novel uses of Magellan® PRP for a variety of surgical applications is being developed as an extension of this disclosure. The therapeutic goal of this approach is the use of PRP in infection prevention in surgical sites. Developing autologous PRP gel that can be readily incorporated with Vancomycin to be used as a stand alone and/or adjunct to other infection prevention strategies in surgical procedures may be envisioned. This example is expected to demonstrate novel use of autologous PRP not only for spine surgeries, but also in other surgical procedures. The results from this example could have immediate effect reducing the cost of post surgical wound infection. This benefit may ultimately translate into a more efficient “localized” antimicrobial coverage allowing for a reduction in the total antibiotic dose used in surgeries.

### Example 4

### Vancomycin-Coupled Bovine PRP Gel

This example serves as a proof of concept for vancomycin-enriched platelet rich plasma (PRP) formula as a potential therapy for wound site infection. The key issue addressed in this study was to determine the feasibility of enrichment of platelet rich plasma for formation of platelet gel without altering the function and handling characteristics of the platelet gel.

Study Design: This example evaluated the appearance and characteristics of antibiotics-enriched bovine PRP. There were two variables in this study: 1) choice of antibiotics, and 2) presence/absence of thrombin. Two antibiotics were used to couple PRP in this study: 1) Vancomycin and 2) Cefazolin. Thrombin, a common reagent for activation of fibrinogen in PRP (for use as a gel would sealant), was used to activate platelet gel-antibiotics/PRP mixture formation, and compared to a mixture not activated by thrombin.

De-ionized water; phosphate buffered saline; calcium chloride (arco orgnaics); Bovine Thrombin (Fisher Scientifics); Bovine Blood (anti-coagulant added); Cefazolin Sodium Salt (Alexis, Fisher Scientific: CAS 27164-46-1); Vancomycin Hydrochloride (Acros Organics, Sigma-Aldrich: CAS 1404-93-9); Arteriocyte Medical System (AMSI) Magellan Autologous Platelet Separatio (APGS) system (AMS100); AMSI Magellan APS disposable kit (AMS305); Centrifuge tubes, pipettors, and other lab wares; scale.

Preparation of Bovine PRP: 60 ml of Bovine blood (anti-coagulant added) was processed with the Magellan® system to obtain 6 ml of bovine PRP. Bovince PRP was stored in a refrigerator.

Preparation of CaCl₂ Solution: CaCl₂ (Calcium Chloride, 96% extra pure, powered, anhydrous, Acros Organics) was dissolved in de-ionized water by ration of 1:10 (weight/volume) at ambient conditions to obtain 10% CaCl₂ solution.

Dissolution of Antibiotics in CaCl₂ solution: Initial target dose of antibiotics was based on body weight and was estimated at 1.5 mg/kg body weight per unit of PRP, or 105 mg for a 70 kg adult. 3 ml PRP was prepared from 60 ml Bovine Blood with the Magellan® System, resulting in a 2× standard concentration. 105 mg was dissolved into 3 mL of saline solution. 1 ml each of PRP Vancomycin/saline solution were combined to yield a product approximating standard PRP, containing desired amount of Vancomycin.

The above solution was further modified with concentration of approximately 75 ug/ml (antibiotic/solution), which was appropriate for local (non-systemic) application:

Vancomycin: To achieve the revised target dose of 75 ug/ml, 5 mg antibiotic was added per 6 ml 10% w/v CaCl₂ solution used to reconstitute thrombin for activation of 6 ml PRP.

5 mg Vancomycin hydrochloride (Acros Organics) was dissolved into 6 ml 10% w/v CaCl₂ solution at ambient conditions for a concentration of 833 ug/ml. Agitation was necessary to properly dissolve Vancomycin due to its large grain sizes.

Cefazolin: 5 mg Cefazolin hydrochloride (Alexis) was dissolved into 6 ml 10% w/v CaCl₂ solution at ambient conditions for a concentration of 833 ug/ml. Slight agitation was necessary to dissolve Cefazolin.

Dissolution of Bovine Thrombin in Antibiotic/10% w/v CaCl₂ solution: Bovine thrombin (10 kU, powder, Fisher Scientific, reconstitute in10 ml D.I. H₂O) was weighed and separated in ten 1 kU units (approximately 110 mg each).

1 kU of bovine thrombin was dissolved into 1 ml of Antibiotic/10% w/v CaCl₂ solution or Vancomycin/10% w/v CaCl₂ solution.

Preparation of Cefazolin- or Vancomycin-Coupled PRP: 0.6 ml of the bovine thrombin/Vancomycin/10% w/v CaCl₂ solution was added to 6 ml of bovine PRP to obtain 6.6 ml of Vancomycin coupled PRP gel to produce an activated gel with a final concentration of ˜75 ug/ml.

For controls, 0.6 ml of the Vancomycin/10% w/v CaCl₂ solution (without thrombin) was added to 6 ml of bovine PRP to obtain 6.6 ml of Vancomycin coupled PRP gel to produce an activated gel with a final concentration of ˜75 ug/ml.

Evaluation of Cefazolin- or Vancomycin-Coupled PRP: After mixing, the antibiotic-enriched PRP with and without thrombin were left on bench top. Visual observation was used to evaluate the appearance and characteristics of the mixture material. This process was conducted under ambient conditions.

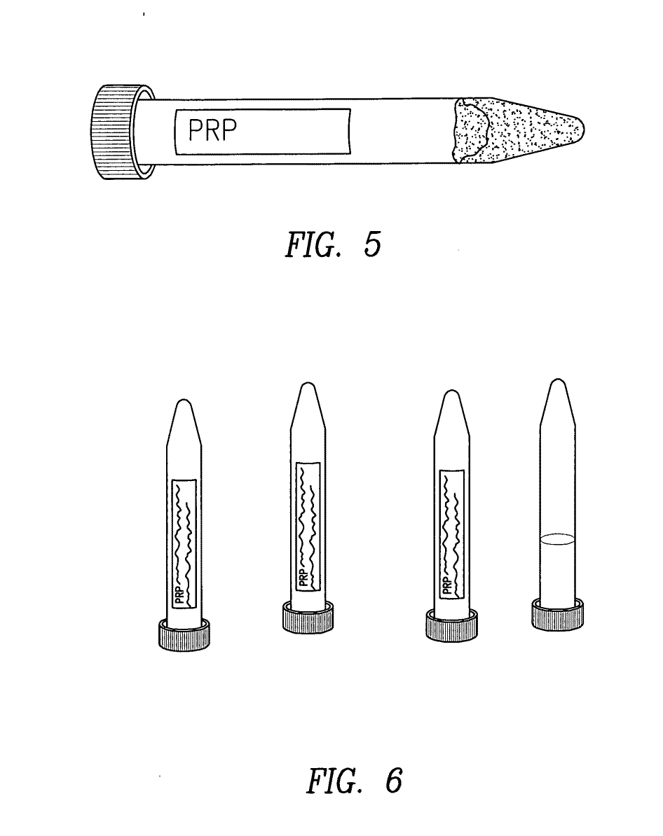

Results: An initial dose of 1.05 g per unit PRP resulted in immediate aggregation into a solid when 1 ml of 2× concentration PRP was combined with the Vancomycin solution (see FIG. 5). No further testing was performed.

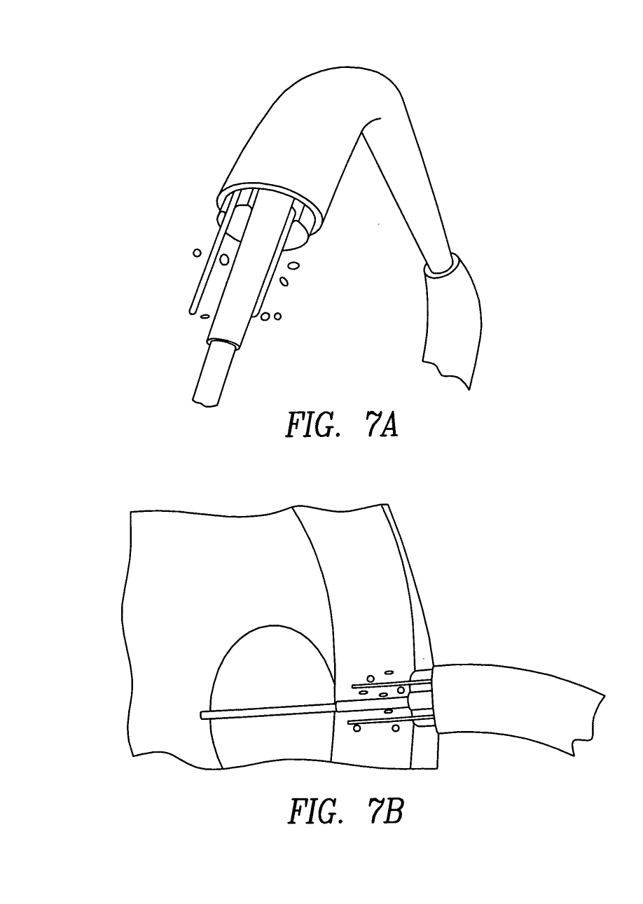

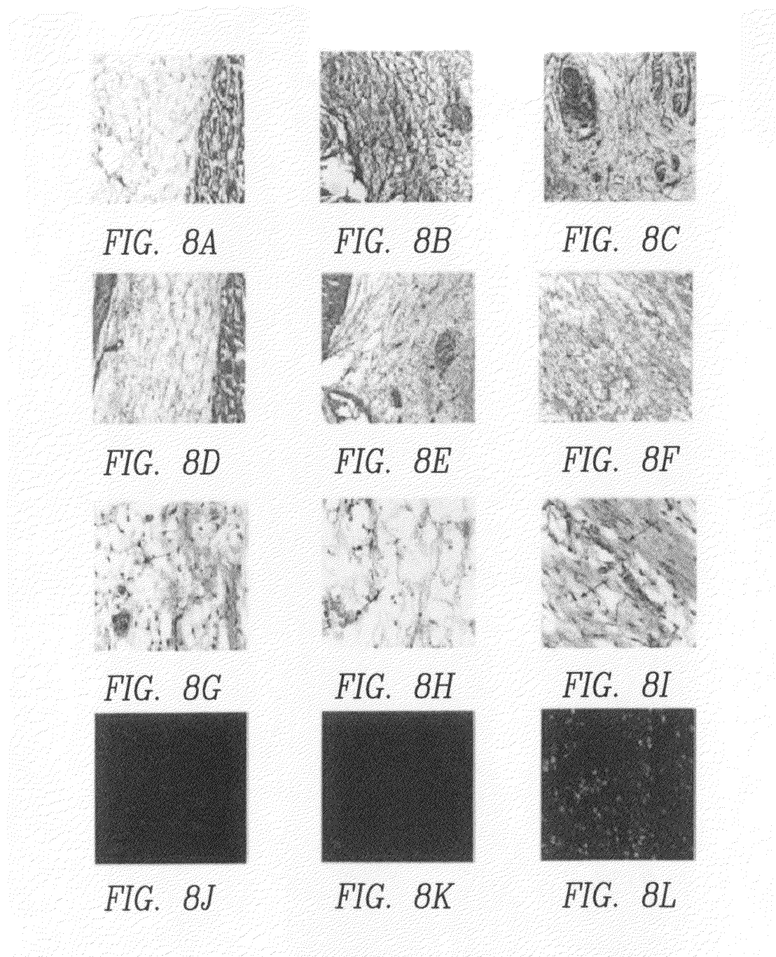

Use of revised preparation protocols and dosing resulted in gel formation with both antibiotic agents. Results are presented below in Table 4-1 and FIG. 6.

TABLE 4-1
Thrombin Activation of Antibiotic-enriched Bovine PRP

| Antibiotic | Thrombin | Observation of PRP/Platelet Gel |
|---|---|---|
| Cefazolin | Yes | Mixture gels immediately within minutes, |
| | | with exudation of liquid of small volume; |
| | | no precipitation or aggregation observed. |
| Cefazolin | No | Mixture does not gel and remain as |
| | | viscous liquid; no precipitation or |
| | | aggregation observed. |
| Vancomycin | Yes | Mixture gels immediately within minutes; |
| | | difficult to observe extent of |
| | | precipitation with gels. |
| Vancomycin | No | Mixture remains as viscous liquid with |
| | | white solid precipitates forming upon |
| | | addition of Vancomycin/CaCl₂ solution. |

The results of this study demonstrated that the addition of large quantities of vancomycin (on the order used in systemic administration) to PRP, even in the absence of thrombin led to rapid formation of heterogenous solids very distinct from gel formation due to activation by thrombin. The aggregates were suspected to be protein aggregations due to significant changes in the chemical environment, but more testing is needed. This would preclude use of PRP to deliver vancomycin in great quantity without additional modification. However, addition of smaller quantities of vancomycin (sufficient for local application) via dissolution into CaCl₂ solution (subsequently used to reconstitute thrombin for PRP activation) did not interfere with or premarturely initiate activation of PRP and gel formation. Similarly, addition of cefazolin to PRP did not appear to alter the properties PRP. In each of these cases, integrity of the PRP gel increased as expected following activation. These findings indicated that Vancomycin and Cefazolin could be used to enrich PRP without compromising PRP's function. The results also suggested that regular, sustained release of the antibiotic agent could be expected during progression of the hemostatic/healing processes initiated by PRP application, providing benefit of local antimicrobial inhibition.

### Example 5

### Repair of a Full-Thickness Cranial Defect with Autologous Platelet Gel

Though calvarial bone autograft remains the gold standard for repair of cranial defects, the amount of graft material is limited, and donor site morbidity remains a pressing concern. Synthetic graft have improved materials have shown promise, but to date are not sufficiently osteo-conductive/-inductive. Large defect repairs are not effectively replaced by bone tissue. Recent advances in osteogenic therapies permit development alternatives cranial repair strategies that do not require a source of graft material for new bone formation.

Table 5-1: Right side defects in group A at each time point was repaired with APG only. Groups B, C, and D was repaired with APG loaded with BMP, MNCs, or both, respectively. In groups 1-3 left side defects was repaired by calvarial bone autograft. In group 4, animals retained intact parietal bone for control Animals were sacrificed and reconstructed crania were recovered either immediately post surgery, 6 or 12 months.

Platelet rich plasma (PRP) can be used to induce bone formation, especially when used in conjunction with osteogenic bone morphogenetic proteins (BMP) and/or marrow-derived multinuclear cells (MNCs). PRP contains high concentrations of chemotactic growth factors that may attract osteoclastic precursors, and has been demonstrated to enhance both osteogenesis and vascular infiltration of regenerating tissue. The Magellan® system can rapidly produce PRP from a small amount of blood, which can be used for wound repair following platelet activation by thrombin. The resulting autologous platelet gel (APG) is already approved for use with autologous bone grafts in orthopedic procedures. In this example use APG as an osteogenic substrate in the established sheep cranial defect model is examined under the hypothesis that it will generate formation of new, organized, vascularized bone within the defect with robust mechanical properties. Furthermore, addition of BMP and MNCs will enhance the rate of osteogenesis, and reduce the time required for new bone formation.

Sheep cranial defect model: Thirty-nine skeletally mature female sheep (including 3 replacements) is randomly assigned to one of eight groups (n=3/each) (Table 5-1). Unilateral or bilateral rectangular defects measuring 1.5×3 cm is created in the parietal bones of each sheep. In animals with bilateral defects (groups 1-3), the right side is repaired with PRP (groups A), PRP+BMP (groups B), PRP MNC (groups C), or PRP+BMP+MNC. The left side is repaired with a full thickness cranial bone autograft. The remaining groups (groups 4) receive similar right side reconstructions, but retain intact left-side parietal bone to serve as controls.

TABLE 5-1

| Reconstruction | | Time of Sacrifice | | |
|---|---|---|---|---|
| Type | Right Side | Post-Surgery | 6 months | 12 months |
| Bilateral | PRP | Group 1A, N = 3 | Group 2A, N = 3 | Group 3A, N = 3 |
| | PRP + BMP | Group 1B, N = 3 | Group 2B, N = 3 | Group 3B, N = 3 |
| | PRP + MNC | Group 1C N = 3 | Group 2C, N = 3 | Group 3C, N = 3 |
| | PRP + BMP + MNC | Group 1D, N = 3 | Group 2D, N = 3 | Group 3D, N = 3 |
| Unilateral | PRP | Not testesd (N/T) | N/T | Group 4A, N = 3 |
| | PRP + BMP | N/T | N/T | Group 4B, N = 3 |
| | PRP + MNC | N/T | N/T | Group 4C, N = 3 |
| | PRP + BMP + MNC | N/T | N/T | Group 4D, N = 3 |

Platelet rich plasma production: 60 mL of blood is drawn from each animal into a 60 mL syringe, immediately before surgery. The syringe is loaded into the Magellan® Autologous Platelet Separator. The Magellan® system is then used to produce 6 ml of PRP for use in the reconstruction. For reconstructions with an MNC component, the Magellan® system is also used to isolate MNCs from the appropriate centrifugation layer. Calcified thrombin is prepared by adding 1,000 units thrombin to each ml of 10% CaCl₂ solution. The solutions is refrigerated until use.

Surgical Procedure: The surgical procedure as described in a previous IACUC approved study is adapted to generate and repair full thickness cranial defects with PRP. Animals are operated upon under general endotracheal anesthesia. Swelling of the brain is controlled by periooperative administration of cephalexin and intraoperative administration of manitol. The defect site is exposed by mid-sagittal incision, and a sub-periosteal dissection is conducted. As previously described, the 4.5 cm² defects is created using a low-speed drill under constant irrigation.

For all PRP reconstructions of cranial defects (right side cranioplasty), a titanium mesh is trimmed to overlap the edges of the defect. The mesh is shaped to fit the contour of the defect, and then secured using micro screws to the superficial surface of the surrounding native bone. Once the desired geometry is obtained, APG is applied to the defect. The Magellan® dual syringe dispenser system is used to activate platelets by mixing PRP with calcified thrombin solution at a 1:10 ratio. In groups B, C and D, thrombin is loaded with BMP-2 and/or MNCs.

The surgical area is kept dry and the APG is placed in the defect and shaped to the ideal contour. The left side defects is repaired with standard calvarial bone autograft. The wound is closed once the cement in APG fully sets (˜30 min.). Prior to sacrifice, high resolution scans are made of the repaired defects. Animals are sacrificed either immediately post-surgery (groups 1), or at 6 (groups 2) or 12 months (groups 3 and 4). Following sacrifice, cranial samples are harvested, and stored at −20° C. for analysis.

Biomechanical testing: The impact tolerance of the PRP/titanium constructs and bone autografts is tested using a standard vertical drop test. A custom made testing apparatus comprising jig, suspended impact mass with rounded indenter (4.76 diameter with 50 mm radius of curvature), load cell and accelerometer is used. Total impact mass is 3.227 kg. The center of the original defect is marked on each cranial specimen. The specimen is positioned under the impactor so that the point of impact is at the mark. The magnetically suspended impact mass is dropped from heights increasing from 19.0 cm, as required to fracture the specimen. A computer based data acquisition system is used to record data from the load cell and accelerometer. Data is acquired for 2.000 s at a sampling frequency of 4000 Hz. All specimens is fixed in 10% formalin for histology analysis immediately following the drop test.

Histology: One sample from each group is histologically evaluated without mechanical testing. The remainder is evaluated following fracture during drop tests. To assess post-surgical osteogenesis in the PRP constructs and autografts, calcein blue, calcein green, tetracycline, xylenol orange, or alizarin red is administered intravenously, beginning immediately post-surgery and continuing until sacrifice. Un-decalcified cranial specimens are trimmed to include both reconstructed areas and surrounding native bone. The specimens is embedded in Spurr's plastic and sectioned. Sections are mounted, hand-polished to a final thickness of ˜50 um. Alternating adjacent sections is stained with hemotoxylin and eosin or left unstained. H&E stained sections is evaluated with standard light microscopy. Unstained sections are evaluated by fluorescence microscopy to estimate deposition rates for new bone in the cement constructs.

Statistical Analysis: For biomechanical testing, statistical analysis is performed with SAS 8.2. 2-factor ANOVA is used evaluate differences between defect repair groups A, B, C, D and time at—0, 6 and 12 months and bone autograft in measurements taken from different specimens. Paired t-tests is used to evaluate differences between measurements taken from specimens from the same skull. A significance level of 0.05 is assumed for all tested variables.

PRP has successfully been used as a template for new bone formation in many cases. It is expected that groups with PRP/BMP and PRP/BMP/MNC reconstructions will yield the most robust tissues after 1 year. However, PRP alone has not been successful in all cases, nor has PRP repair been implemented in this defect model; it is possible that there may not be sufficient osteogenesis, particularly in the PRP only reconstructions. To minimize this risk, the procedure is tested first on a small “pilot” subset of animals from each repair type A, B, C and D to confirm efficacy. If bone generation is found unacceptable in only the PRP case, these experimental groups (1A-4A) is dropped from the study.

Additionally, though the titanium mesh is expected to provide adequate impact resistance throughout regardless of bone formation, it is possible APG layered over the mesh at time 0 may not have adequate stiffness for effective drop testing. If this is case, alternative methods (compression testing) will be used to characterize the properties reconstruction.

If successful, PRP/BMP reconstructions may represent a significant leap forward for craniofacial surgical methods, which eliminates the need for cranial bone autograft and the associated donor site morbidity. It may have additional implications for other bone defect repair methods in which a significant amount of osteoinductive/osteoconductive graft material is required.

Deliverables include: PRP/titanium mesh full thickness cranial defect technique; mechanical characterization of PRP reconstructions over time; month-by-month analysis of osteogenesis in each repair technique; and optimal requirements for robust osteogenesis with APG (BMP, MNC additives).

### Example 6

### Improved Cell Survival in Infaracted Myocardium Using a Novel Combination Transmyocardial Laser and Cell Delivery System

After ischemic cardiac disease, stem cell therapy has had limited success due to inadequate microvascular environment in order survive once implanted into scar tissue. The goal of this investigation was to create a microvascular environment into infarcted myocardial tissue using transmyocardial laser revascularization (TMR) as a pretreatment before cell implantation, and evaluate cell survival afterwards. TMR is a process by which channels are created into ischemic myocardium by either laser or mechanical means to enhance myocardial perfusion by directly supplying tissue with endoventricular oxygenated blood or angiogenesis. In animals, TMR is known to upregulate VEGF, bFGF along with other growth factors, and induce at least a limited degree of angiogenesis using either mechanical or laser treatment. This experiment is performed to test whether using TMR as pretreatment to cell therapy may improve the microenvironment to enhance cell retention and long-term engraftment in a large-animal model of myocardial infarction (MI). TMR may act synergistically with the paracrine factors secreted by the engrafted mesenchymal stem cells (MSCs), leading to a more potent effect on myocardial remodeling following an MI. The use of TMR intramyocardially, when cell injections are delivered concurrently or adjuctively, may release pro-thrombin into the tissue, which may activate the platelet content resulting in greater retention and survival of the heterogenic injected cells. In addition, TMR also may increase angiogenic growth factors in the laser injury site, thus providing a viable environment for the stem cells to survive and home better, resulting in higher retention and survival of intramuscularly injected stem cells after TMR. In this example, a novel cell delivery system, which was used to allow for radial dispersion of cells around the laser channel, is also described (FIGS. 7 A-B).

Study Design and Methods: Cell Lines: Porcine domestice mesenchymal stem cells (MSC) were used in an allogeneic fashion, without the need for bone marrow harvesting in each animal subjected to the infarction protocol. The cells were expanded in 10% FBD a-MEM media up to 80% confluence, and were used up to passage 8. One the day of the surgery, the cells were trypsinized, washed with PBS, and labeled with 5 um of either 5-chloronethylfluorescein (CMFDA-green) for the TMR+Cells group (Molecular Probes, Invitrogen). In three experiments, a sample of the cells was replated to check for cell viability; at 3 weeks the cells were re-suspended and the presence of the fluorescent dye confirmed.

Echocardiography: Transthoracic echocardiograms were performed at baseline, prior to cell injection (2 weeks post-MI), and prior to sacrifice (3 weeks post-MI and 1 week post-cell injection) to evaluate LV function and rule out any potential adverse effects using a Toshiba Aplio platform (SSA-770A) with tissue Doppler (TD) capabilities and a 3.0 MHz transducer. Short- and long-axis views were obtained from a right parasternal approach, and four- and two-chamber views were obtained from the apex. Wall and cavity dimensions were determined from short-axis views at the level of papillary muscle, allowing for calculation of fractional area shortening. The apical views were used to calculate cavity volumes and the ejection fraction (EF) offline using a modified Simpson approach. For each measurement, three consecutive cardiac cycles were traced and averaged by an experienced examiner in a blinded fashion.

Porcine Model of Myocardial Infarct: Adult crossbred domestic pigs (n=6) were used for this study. Under general anesthesia, intubated and ventilated with room air supplemented with oxygen and isoflurane (0.5-2%), the animals underwent a percutaneous lateral wall myocardial infacrtion by inflating an angioplasty balloon in the proximal circumflex artery for 60 min. Cell implantation was performed after allowing the animals to recover for 2 weeks and let the infarct mature. The pigs were again sedated, intubated, and placed on general anesthesia. The lateral wall of the left ventricle was exposed using a left thoracotomy. The general area of the scar was identified by visual inspection and preoperative echocardiography. The scar region was divided in three linear fields along the obtuse marginal branches of the circumflex. Each field received four treatments, spaced approximately 2 cm apart (TMR+Cells, TMR alone, and Cells alone), using the Phoenix™ (Cardiogenesis, Irvine, Calif.). For each injection on average 2 million fluorescently labeled cells were used (same number per injection in each animal for TMR+Cells vs. Cell-alone groups). For the Cells-alone group, the same hand piece was used as for the other groups, after flushing the residual cells and cutting the distal laser fiber optic tip. Each of the injection sites was marked with a different type of suture, depending on treatment allocation. The third stage was accomplished at 1 week following the stem cell implantation. The animals were sacrificed after echocardiography and the hearts collected for analysis.

Histology: Transmyocardial core samples of myocardium center on the axis of the TMR treatment were obtained from each animal (4×3 tissue blocks per animal and sectioned into 10-um slices). The tissue was processed for frozen sections after dehydration in sucrose and liquid nitrogen freezing. Sections were stained with hematoxylin and eosin or Masson's trichrome. A set of samples was also processed using paraffin embedding and sectioning. Quantification of the surviving cells at 1 week postimplantation was done by counting the number of fluorescent cells per high-power filed (0.2 mm²), by two blinded observers in a total of 300 high-power fields. The mean number of number of cells per field was used for analysis.

Statistical Analysis: Data are presented as mean±SD. Continuous data were analyzed by ANOVA, when the ANOVA F-value was significant.

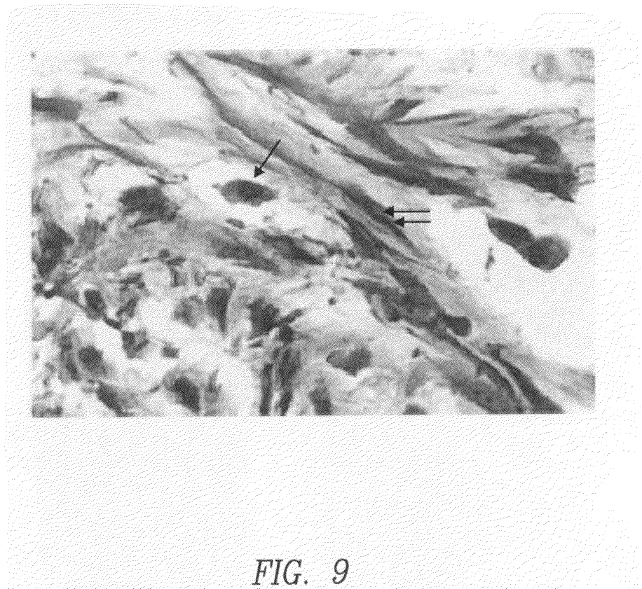

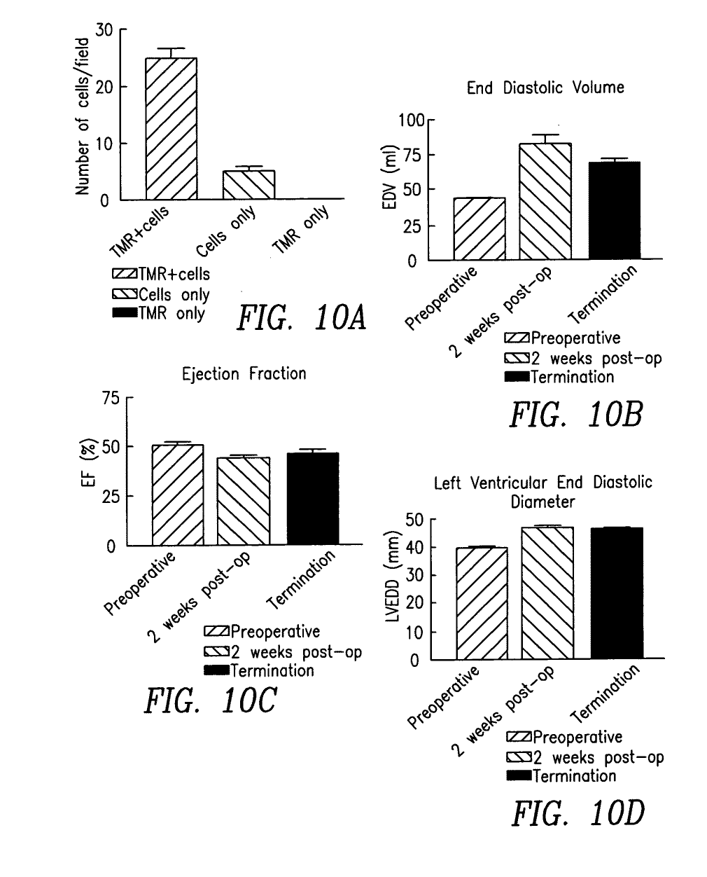

Results: All animals survived to the end of the study. One animal had an episode of ventricular tachycardia during balloon inflation, resulting in balloon dislodgement and a very small infarct; this was subsequently due to the lack significant scar on the histological and clinical analysis. The histological findings are depicted in FIG. 8. Three weeks following circumflex occlusion a significant subendocardial scar formed in the postero-lateral wall. The extent of fibrosis in the three subgroups is depicted in FIGS. 8A-C. FIGS. 8D-I demonstrate hematoxylin and eosin staining of myocardium 3 weeks after infarct, 1 week posttreatment [(FIG. 8D) Cells, (FIG. 8E) TMR, (FIG. 8F) TMR+Cells; (FIG. 8G, 8H, 8I) are higher magnification images of (FIGS. 8D, 8E and 8F) respectively]. There was increased cellularity (FIGS. 8G and 8I) of characteristic mesenchymal cells versus the round possible inflammatory cells (FIG. 8H). FIG. 9 depicts a high-magnification image of a section for the TMR+Cells group showing both characteristic mesenchymal and inflammatory cells. FIGS. 8J-L demonstrate the fluorescent imaging used to evaluate cell survival in the three treatment arms, suggesting increased cell survival in the TMR+Cell (green) over the Cells-only (red) or TMR alone group. This was confirmed by quantification of cell survival, depicted in FIG. 10A, showing that the TMR+Cells had 25±5 cells per high power field evaluated compared to only 5±2 cells in the Cells-only field.

Echocardiographic findings are shown FIG. 10B-D for all the animals. No significant pericardial effusions were seen following the procedure. The scar formation on the lateral wall of the heart in the experimental model led to only a small insignificant reduction in the EF in the circumflex artery distribution. However, it did lead to excellent animal survival while conducting the study. Left ventricular remodeling was observed by 2 weeks with increased end diastolic volume LVED volumes; 1 week later there was a trend towards a reduction in the ventricular dimensions following TMR+Cells treatment (p=0.3). Also, the end diastolic diameter increased after infarction but actually decreased 1 week after treatment at termination.

Conclusion: This study indicates improved cell survival when the MSCs are delivered in combination with laser TMR following an MI in a clinically relevant large-animal.

It is understood that modifications which do not substantially affect the activity of the various embodiments of this disclosure are also provided within the definition of the disclosure provided herein. Accordingly, the following examples are intended to illustrate but not limit the present disclosure. While the claimed disclosure has been described in detail and with reference to specific embodiments thereof, it will be apparent to one of ordinary skill in the art that various changes and modifications can be made to the claimed disclosure without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this disclosure, and are covered by the following claims.

### REFERENCES

Centers for Disease Control and Prevention, N.C.f.H.S.V.a.H.S., Vol 127. Hyattsville (MD): Department of Health and Human Services; (1997).
Haley R W, C. D., White J W, Morgan W M, Emori T G. Am J Epidemiol 159-167. (1985).
R L., N. Am j Med 104, 11s-16s (1998).
Green J W, W. R., Ann Surg, 264-268 (1977).
Taylor G J, M. F., Moses H W, Dove J T, Katholi R E, Malik S A., Am J Cardiol 65, 309-313 (1990).
Ofluoglu, E. A., et al., Arch Orthop Trauma Surg 127, 391-396 (2007).
Rosenberg, A. D., et al., J Bone Joint Surg Am 90, 226-232 (2008).
Fallon, M. T., Journal of Surgical Research 86, 97-102 (1999).
Poelstra, K. A., et al., J Biomed Mater Res 60, 206-215 (2002).
Vodna, L., Bubenikova, S. & Bakos, D., Macromol Biosci 7, 629-634 (2007).
Allen, D. M., Ann Acad Med Singapore 26, 687-690 (1997).
Azzam, R., Hamade, N., Bedrossian, N. & Assaad, C., J Med Liban 50, 211-215 (2002).
Carrau, R. L., et al., Head Neck 13, 311-317 (1991).
Kato, Y., et al., Crit. Care Med 35, 1763-1768 (2007).
Labbe, A. C., et al., Infect Control Hosp Epidemiol 24, 591-595 (2003).
Yeaman, M. R., Clin Infect Dis 25, 951-968; quiz 969-970 (1997).
Vang, S, N., et al., J Extra Corpor Technol 39, 31-38 (2007).
Christensen, K., et al., J Extra Corpor Technol 38, 249-253 (2006).
Jung, F., Bach, R., Mrowietz, C., Seyfert, U. & Franke, R. P., Biomed Tech (Berl) 46, 200-206 (2001).
Rosolski, T., Matthey, T., Frick, U. & Hachenberg, T., Int J Artif Organs 21, 820-824 (1998).
Palumbo, M., Valdes, M., Robertson, A., Sheikh, S. & Lucas, P., Spine J 4, 287-292 (2004).
Barie, P. S., World J Surg 22, 118-126 (1998).
Chambers, S. T., Intern Med J 35 Suppl 2, S63-71 (2005).
Debarge, R., Nicolle, M. C., Pinaroli, A., Ait Si Selmi, T. & Neyret, P., Rev Chir Orthop Reparatrice Appar Mot 93, 582-587 (2007).
Cordero-Ampuero, J., Esteban, J., Garcia-Cimbrelo, E., Munuera, L. & Escobar, R., Acta Orthop 78, 511-519 (2007).
Frommelt, L., Injury 37 Suppl 2, S87-94 (2006).
Ha, K. Y., Chung, Y. G. & Ryoo, S. J., Spine 30, 38-43 (2005).
Nishimura, S., Tsurumoto, T., Yonekura, A., Adachi, K. & Shindo, H., J Orthop Sci 11, 46-50 (2006).
Rao, N. & Regalia, D. M., Clin Orthop Relat Res 451, 34-37 (2006).
Schmidt, A. H. & Swiontkowski, M. F., J Am Acad Orthop Surg 8, 285-291 (2000).
Vlastarakos, P. V., Nikolopoulos, T. P., Maragoudakis, P., Tzagaroulakis, A. & Ferekidis, E., Laryngoscope 117, 668-673 (2007).
Poelstra, K. A., Barekzi, N. A., Grainger, D. W., Gristina, A. G. & Schuler, T. C. Spine 25, 406-410 (2000).
Klezl, Z., et al. Acta Chir Orthop Traumatol Cech 74, 305-317 (2007).
Bratzler, D. W., et al. Arch Surg 140, 174-182 (2005).
Zhou, T., et al. Electrophoresis 27, 1790-1796 (2006).
Tigani, D., et al. Journal of materials science 19, 1325-1334 (2008).
Dawson, S., et al., Oncol Rep 7, 1239-1242 (2000).
Hawn, M. T., et al., J Am Coll Surg 203, 803-811 (2006).
Horner-Vanniasinkam, S., Int J Infect Dis 11 Suppl 1, S17-22 (2007).
Harrison, P. & Cramer, E. M., Blood Rev 7, 52-62 (1993).
Waters, J. H. & Roberts, K. C., J Extra Corpor Technol 36, 250-254 (2004).
Cody C. Trowbridge, M., CCP; Alfred H. Stammers, MSA, CCP; Edward Woods, MD; Bianca R. Yen, BS, CCP; Myra Klayman, BS, CCP; Christian Gilbert, MD., JECT. 37, 381-386 (2005).
Witsø E, P. L., Loseth K, Benum P, Bergh K., Acta Orthop Scand 71, 80-84 (2000).
Stallmann, H. P., de Roo, R., Faber, C., Amerongen, A. V. & Wuisman, P. I., J Orthop Res 26, 531-538 (2008).
Stallmann, H. P., et al., Peptides 26, 2355-2359 (2005).
Faber, C., et al., Antimicrob Agents Chemother 49, 2438-2444 (2005).
Dijkshoorn, L., et al., Antimicrob Agents Chemother 48, 4919-4921 (2004).
Lupetti, A., et al., J Antimicrob Chemother 54, 603-608 (2004).
Stallmann, H. P., Faber, C., Bronckers, A. L., Nieuw Amerongen, A. V. & Wuisman, P. I., J Antimicrob Chemother 54, 472-476 (2004).
Nibbering, P. H., et al., J Nucl Med 45, 321-326 (2004).
Stallmann, H. P., et al., J Antimicrob Chemother 52, 853-855 (2003).
Lupetti, A., et al., Antimicrob Agents Chemother 47, 262-267 (2003).
Lupetti, A., et al., Antimicrob Agents Chemother 46, 1634-1639 (2002).
Lupetti, A., Welling, M. M., Mazzi, U., Nibbering, P. H. & Pauwels, E. K., Eur J Nucl Med Mol Imaging 29, 674-679 (2002).
C J DeFrances, K A Cullen, and L J Kozak, National Hospital Discharge Survey: 2005 Annual National Center for Health Statistics (2007).
D. S. Young, B. S. Sachais, and L. C. Jefferies, Clin Chem, 46(7): p. 955-66 (2000).
J. M. Lane, E. Tomin, and M. P. Bostrom, Clin Orthop Relat Res, (367 Suppl): p. S107-17 (1999).
Frost and Sullivan, US Bone Graft and Bone Substitutes. 2007: Palo Alto, Calif.
I. T. Jackson and R. Yavuzer, Br J Plast Surg, 2000. 53(1): p. 24-9 (2000).
P. W. Hitchon, et al., J Neurosurg, 95(2 Suppl): p. 215-20 (2001).
B. R. Constantz, et al., Science, 267(5205): p. 1796-9 (1995).
P. D. Costantino, et al., Hydroxyapatite cement. I. Arch Otolaryngol Head Neck Surg, 117(4): p. 379-84 (1991).
C. D. Friedman, et al., Arch Otolaryngol Head Neck Surg, 117(4): p. 385-9 (1991).
R. V. Silva, et al., Int J Oral Maxillofac Surg, 34(2): p. 178-84 (2005).
A. J. Ambard and L. Mueninghoff, J Prosthodont, 15(5): p. 321-8 (2006).
S. Rupprecht, et al., J Craniomaxillofac Surg, 31(3): p. 149-53 (2003).
D. Lew, et al., J Oral Maxillofac Surg, 55(12): p. 1441-9; discussion 1449-51 (1997).
S. B. Baker, et al., Plast Reconstr Surg, 109(6): p. 1789-96 (2002).
S. R. Durham, J. G. McComb, and M. L. Levy, Neurosurgery, 52(4): p. 842-5; discussion 845 (2003).
D. B. Matic and P. N. Manson, J Craniofac Surg, 15(3): p. 415-22; discussion 422-3 (2004).
R. Verheggen and H. A. Merten, Acta Neurochir (Wien), 143(9): p. 919-26 (2001).
D. J. Verret, et al., Otolaryngol Head Neck Surg, 133(6): p. 897-9 (2005).
J. E. Zins, A. Moreira-Gonzalez, and F. A. Papay, Plast Reconstr Surg, 120(5): p. 1332-42 (2007).
G. X. Ni, et al., Biomaterials, 27(9): p. 1963-70 (2006).
J. E. Losee, et al., J Craniofac Surg, 14(1): p. 117-24 (2003).
P. A. Chambers, R. A. Loukota, and A. S. High, Br J Oral Maxillofac Surg, 45(1): p. 77-8 (2007).
A. Elshahat, et al., J Craniofac Surg, 15(3): p. 483-9 (2004).
H. G. Moghadam, et al., J Oral Maxillofac Surg, 62(2): p. 202-13 (2004).
R. D. Hayward, Br J Neurosurg, 13(5): p. 490-1 (1999).
D. Simpson, Neurosurgery, 26(2): p. 361 (1990).
D. Simpson, J Neurosurg, 22: p. 292-3 (1965).
N. Fushiki, Nippon Geka Hokan, 37(3): p. 413-9 (1968).
D. A. Koppel, K. F. Moos, and F. S. Walker, Br J Oral Maxillofac Surg, 37(1): p. 70-2 (1999).
Y. Ducic, J Oral Maxillofac Surg, 60(3): p. 272-6 (2002).
J. A. Jansen, et al., J Control Release, 101(1-3): p. 127-36 (2005).
A. Maniker, S. Cantrell, and C. Vaicys, Neurosurgery, 43(4): p. 953-4; discussion 955 (1998).
J. Wiltfang, et al., J Oral Maxillofac Surg, 62(1): p. 29-35 (2004).
J. Wiltfang, et al., Clin Oral Implants Res, 15(2): p. 187-93 (2004).
K. Akca, et al., Int J Periodontics Restorative Dent, 27(4): p. 387-92 (2007).
T. M. You, et al., Oral Surg Oral Med Oral Pathol Oral Radiol Endod, 103(4): p. e8-12 (2007).
T. M. You, et al., Int J Oral Maxillofac Implants, 22(3): p. 417-22 (2007).
D. Nikolidakis, et al., Tissue Eng, 12(9): p. 2555-63 (2006).
K. M. Hansson, et al., Biomaterials, 26(8): p. 861-72 (2005).
T. B. Jensen, et al., An experimental study in dogs. J Orthop Res, 22(3): p. 653-8 (2004).
G. Weibrich, et al., Bone, 34(4): p. 665-71 (2004).
S. Fontana, et al., Implant Dent, 13(1): p. 73-8 (2004).
K. A. Schlegel, et al., Biomaterials, 25(23): p. 5387-93 (2004).
W. Zechner, et al., Int J Oral Maxillofac Implants, 18(1): p. 15-22 (2003).
W. J. Berghoff, W. S. Pietrzak, and R. D. Rhodes, Orthopedics, 29(7): p. 590-8.
P. A. Everts, et al., Acta Anaesthesiol Scand, 50(5): p. 593-9 (2006).
M. Wrotniak, T. Bielecki, and T. S. Gazdzik, Ortop Traumatol Rehabil, 9(3): p. 227-38 (2007).
A. Thor, et al., Int J Oral Maxillofac Surg, 36(12): p. 1164-71 (2007).
K. Kinoshita, et al., J Craniofac Surg, 19(1): p. 80-7
J. Slapnicka, et al., J Oral Maxillofac Surg, 66(2): p. 297-301 (2008).
K. Yokota, et al., Acta Orthop, 79(1): p. 106-10 (2008).
S. S. Lin, et al., Conf Proc IEEE Eng Med Biol Soc, 1: p. 4358-61 (2006).
A. Tomoyasu, et al., Biochem Biophys Res Commun, 361(1): p. 62-7 (2007).
Y. Yamada, et al., Cell Transplant, 13(4): p. 343-55 (2004).

What is claimed:

1. A method for treating ischemic cardiac disease with stem cells, comprising:
administering transmyocardial laser revascularization (“TMR”) to an area of infarcted myocardial tissue to create a microvascular environment therein;
implantation of a plurality of stem cells into said area of infarcted myocardial tissue, wherein
the stem cells are isolated from the group consisting of: placenta, adipose tissue, lung, bone marrow, or blood.

2. The method according to claim 1, wherein TMR administered to said myocardial tissue leads to release of prothrombin or thrombin into said infarcted myocardial tissue.

3. The method according to claim 1, wherein said TMR comprises creating small channels in the area of infarcted myocardial tissue.

4. The method according to claim 3, wherein administration of said TMR increases angiogenic growth factors in said area of infarcted myocardial tissue.

5. The method according to claim 4, wherein said angiogenic growth factors influence the survival of said intramuscularly injected stem cells after administration of said TMR.

6. The method according to claim 1, wherein said stem cells are mesenchymal stem cells or hematopoietic stem cells.

7. The method according to claim 1, wherein said stem cells are prepared by a stem cell fractionation process to concentrate the mononucleated cells from the bone marrow.

8. A method to provide a sustained or continuous release of antibiotics directly to an open wound site to prevent or fight infection comprising:
providing a mixture of a predetermined amount of autologous platelet rich plasma (PRP) and providing a predetermined amount of at least one general antibiotic;
wherein the combination of said autologous platelet rich plasma (“PRP”) and said general antibiotic enables sustained or continuous release of the antibiotic.

9. The method according to claim 8, wherein the antibiotic combined with PRP is selected from the group consisting of: adriamycin, erythromycin, gentimycin, penicillin, tobramycin, vancomycin, cefazolin, amoxicillin, cephalosporins, macrolides, fluoroquinolones, sulfonamides, tetracyclines and aminoglycosides.

10. The method according to claim 8, wherein a lower dose of antibiotic is delivered directly at an infection site thus reducing side effects associated with a higher dose systemic delivery; wherein the method is configured to apply the PRP-antibiotic directly to a wound site prior to closing of a wound or an incision.

11. The method according to claim 8, wherein the method is configured to controllably release delivery system for the antibiotics using a multiple-site implant device.

12. A method for stimulating new bone formation in bone defects comprising:
applying an autologous platelet gel and a plurality of mononuclear cells, multinuclear cells, and/or bone morphogenetic proteins to an area of bone defect,
wherein the composition of the autologous platelet gel and the mononuclear cells, multinuclear cells, and/or bone morphogenetic proteins stimulates new bone formation in said bone defect.

13. The method according to claim 12, wherein the multinuclear cells are bone-marrow derived osteoclasts.

14. The method according to claim 12, wherein the bone morphogenic protein is selected from the group consisting of: BMP1, BMP2, BMP3, BMP4, BMP5, BMP6, BMP7, BMP8a, BMP8b, BMP10, and BMP15.

15. The method according to claim 12, wherein the mononuclear cells are selected from the group consisting of: bone-marrow derived mesenchymal stem cells (BM-MSCs) and bone-marrow derived mononuclear cells (BM-MNCs).

16. A method for preparing a stem cell therapeutic composition, comprising the steps of:
obtaining stem cells from a patient;
combining the stem cells with a biological agent to form a stem cell therapeutic composition.

17. The method according to claim 16, wherein the biological agent is selected from a group consisting of: analgesic compounds, antibacterial compounds, including bactericidal and bacteriostatic compounds, antibiotics, antifungal compounds, anti-inflammatories, antiparasitic compounds, antiviral compounds, enzymes, enzyme inhibitors, glycoproteins, growth factors, hormones, steroids, glucocorticosteroids, immunomodulators, immunoglobulins, minerals, neuroleptics, proteins, peptides, lipoproteins, tumoricidal compounds, tumorstatic compounds, toxins and vitamins, or fragments, portions, derivatives, or analogues thereof.

18. The method according to claim 17, wherein antibiotics are selected from a group consisting of: adriamycin, erythromycin, gentimycin, penicillin, tobramycin, vancomycin, cefazolin, amoxicillin, cephalosporins, macrolides, fluoroquinolones, sulfonamides, tetracyclines and aminoglycosides.

19. The method according to claim 17, wherein the stem cell therapeutic composition may be administered to a mammal for treatment of medical conditions, selected from the group consisting of: cardiovascular, thoracic, transplantation, head and neck, oral, gastrointestinal, orthopedic, neurosurgical, and plastic surgery.
